(19) United States
(12) Patent Application Publication
(10) Pub. No.: US 2024/0138888 A1
(43) Pub. Date: May 2, 2024

(54) ORTHOPEDIC IMPLANT FOR BONE REALIGNMENT

(71) Applicant: BioMedtrix, LLC, Whippany, NJ (US)

(72) Inventors: John Brajkovich, Caledon Village (CA); Gregory Thomas Van Der Meulen, Ketchum, ID (US); Christopher G. Sidebotham, Mendham, NJ (US); Christopher Preucil, Ketchum, ID (US)

(73) Assignee: BioMedtrix, LLC, Whippany, NJ (US)

(21) Appl. No.: 18/547,068

(22) PCT Filed: Feb. 25, 2022
(86) PCT No.: PCT/US2022/017903

§ 371 Date: Aug. 18, 2023

Related U.S. Application Data
(60) Provisional application No. 63/153,891, filed on Feb. 25, 2021.

(51) Int. Cl. A61B 17/80; A61B 17/68

(52) U.S. Cl. CPC A61B 17/8028; A61B 2017/681

(57) ABSTRACT

An orthopedic spacer assembly can include a bone plate and a spacer. The bone plate can include a first end portion and a second end portion, the second end portion defining a plurality of anchor openings. The spacer can have a c-shaped body and can include a central bore extending through a thickness of the spacer. The spacer can be disposed between the bone plate and a subject's bone such that the central bore of the spacer aligns with an anchor opening of the plurality of anchor openings.

### CROSS REFERENCE TO RELATED APPLICATION

This application claims the benefit of U.S. Provisional Application Ser. No. 63/153,891, filed on Feb. 25, 2021, which is incorporated by reference herein in its entirety.

### FIELD

This application pertains to implantable orthopedic devices for bone realignment and osteotomy procedures.

### BACKGROUND

In human and veterinary orthopedics, osteotomies of long bones (e.g., the femora, tibiae, and fibulae of the legs and the humeri, radii, and ulnae of the arms) are performed to realign the limb for improved biomechanics Many cases can be realigned in a single plane using a bone plate and screws. However, in some cases additional alignment in another plane can be indicated. In cases where alignment in multiple planes is required, typically a stepped plate is used to provide correction. However, stepped plates can be costly to produce as they are typically machined from solid blocks of material. Moreover, stepped plates with various dimensions in both left and right configurations must be provided such that an appropriately sized implant can be selected, requiring clinicians to maintain large inventories. For example, step sizes may vary between 3 mm and 6 mm in 1 mm increments. Additionally, certain subjects may have anatomy that requires specialty or custom plates. Accordingly, there is a continuing need for improved orthopedic implants to address multiple plane corrections.

### SUMMARY

In a representative embodiment, an orthopedic spacer assembly can include a bone plate including a first end portion and a second end portion, the second end portion defining a plurality of anchor openings, and a spacer. The spacer can have a c-shaped body and comprise a central bore extending through a thickness of the spacer. The spacer can be configured to be disposed between the bone plate and a subject's bone such that the central bore of the spacer aligns with an anchor opening of the plurality of anchor openings.

In some or all embodiments, the spacer comprises a first surface and a second surface separated by a sidewall, and wherein the first surface has a convex shape. In some or all embodiments, the side wall is circular.

In some or all embodiments, the sidewall comprises a slot extending radially inwardly toward the central bore, wherein the slot is configured to engage a positioning tool configured to position the spacer between the bone plate and the second bone portion. In some or all embodiments, the slot is a first slot, and the side wall further comprises a second slot circumferentially offset from the first slot around the body of the spacer.

In some or all embodiments, the first surface of the spacer is configured to be positioned adjacent the bone plate and wherein the second surface is configured to be positioned against a bone portion.

In some or all embodiments, the central bore tapers from a first opening having a first diameter to a second opening having a second diameter greater than the first diameter.

In some or all embodiments, the spacer comprises an opening extending from the central bore to a radially outer edge of the spacer.

In some or all embodiments, a thickness of the spacer is between 1 mm and 10 mm

In some or all embodiments, the spacer is a first spacer and the assembly further comprises a second spacer configured to be disposed between the bone plate and the subject's bone such that the central bore of the second spacer aligns with another of the anchor openings of the plurality of anchor openings.

In some or all embodiments, the spacer is configured to be disposed at least partially around a fastener extending through the anchor opening.

In some or all embodiments, a second surface of the spacer comprises one or more cutouts.

In a representative embodiment, an orthopedic spacer can comprise a curved main body having a longitudinal axis. The main body can have a convex first surface and a second surface on the opposite side of the main body from the first surface along the longitudinal axis, and a first slot extending longitudinally through a thickness of the main body, and extending radially inwardly from an outer perimeter of the main body to a center of the main body such that the main body is C-shaped.

In some or all embodiments, the main body further comprises a sidewall between the first surface and the second surface, and the sidewall comprises a second slot extending radially inwardly from an outer surface of the sidewall.

In some or all embodiments, the sidewall further comprises a third slot circumferentially offset from the second slot around the main body of the spacer from the second slot.

In some or all embodiments, the main body comprises chamfered surfaces at an open end of the first slot.

In some or all embodiments, a closed end portion of the first slot at the center of the main body is countersunk.

In a representative embodiment, a method can include creating an osteotomy in a long bone to create a first bone portion and a second bone portion, securing a first end portion of a bone plate to the first bone portion, and offsetting the first and second bone portions such that the first and second bone portions are in an offset position relative to one another. The method can further include disposing one or more spacers between a second end portion of the bone plate and the second bone portion, and securing the first and second bone portions in the offset position using one or more fasteners.

In some or all embodiments, offsetting the first and second bone portions from one another comprises inserting a sizing implement between a second end portion of the bone plate and the second bone portion.

In some or all embodiments, disposing one or more spacers between the bone plate and the second bone portion comprises mounting a spacer on a positioning implement, and positioning the spacer around a fastener extending through the bone plate and into the second bone portion using the positioning implement.

The foregoing and other objects, features, and advantages of the disclosed technology will become more apparent from the following detailed description, which proceeds with reference to the accompanying figures.

### BRIEF DESCRIPTION OF THE DRAWINGS

FIGS. 1A-1C illustrate a representative repair of a ruptured cranial cruciate ligament by performing a TPLO.

FIG. 2 is a perspective view of a representative embodiment of a bone plate.

FIG. 3 is a side elevation view of the bone plate of FIG. 2

FIG. 4 is a plan view of a representative embodiment of a spacer.

FIG. 5 is a cross-sectional perspective view of the spacer of FIG. 4.

FIG. 6 is a perspective view of a spacer assembly including the bone plate of FIG. 2 and the spacer of FIG. 4.

FIG. 7 is a perspective view of the spacer assembly of FIG. 6.

FIG. 8 illustrates medial patellar luxation.

FIG. 9 illustrates a medialized osteotomy.

FIG. 10 illustrates the spacer assembly of FIG. 6 used in a medialized osteotomy for TPLO.

FIG. 11 illustrates a prior art stepped plate.

FIG. 12 illustrates a perspective view of a representative embodiment of a spacer assembly using in a SHO.

FIGS. 13A-13C illustrate a representative femoral neck angle correction procedure.

FIGS. 14A-14C illustrate a representative femoral version correction procedure.

FIGS. 15A-15C illustrate medial and lateral patellar luxation.

FIGS. 16A-16C illustrate a representative tubercle transposition.

FIGS. 17A-17B illustrate a representative triple pelvic osteotomy procedure.

FIG. 18 is a top down view of a representative example of a spacer.

FIG. 19 is a perspective view of the spacer of FIG. 18 disposed on an exemplary fastener.

FIG. 20 is a perspective view of a representative spacer assembly including the spacer of FIG. 18 coupled to a native tibia for a TPLO.

FIG. 21 is a top down view of an exemplary spacer kit assembly.

FIGS. 22-24 illustrate an exemplary method of implanting the spacer assembly of FIG. 20 in a TPLO procedure.

FIG. 25 is a perspective view of the spacer assembly of FIG. 20 used in a TPLO procedure with the bone plate removed for purposes of illustration.

FIG. 26 is another perspective view of the spacer assembly of FIG. 20 used in a TPLO procedure.

FIGS. 27A-27C illustrate various views of another exemplary embodiment of a spacer.

FIGS. 28A-28C illustrate various views of another exemplary embodiment of a spacer.

FIGS. 29A-29D illustrate various views of yet another exemplary embodiment of a spacer.

FIGS. 30A-30C illustrate various views of still another exemplary embodiment of a spacer.

FIGS. 31A-31C illustrate various views of an exemplary embodiment of a spacer.

FIG. 32 is a side elevation view of one of the sizing implements shown in FIG. 21.

FIG. 33 is a perspective view of the spacer embodiment of FIG. 29A including a slot.

FIGS. 34-35 are perspective views of the spacer of FIG. 18.

FIG. 36 is a top plan view of the spacer of FIG. 18.

FIG. 37 is a bottom plan view of the spacer of FIG. 18.

FIG. 38 is a side elevational view of the spacer of FIG. 18.

FIG. 39 is a front elevational view of the spacer of FIG. 18.

FIG. 40 is a back elevational view of the spacer of FIG. 18.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “cranial” refers to a direction toward the head of the patient and the term “caudal” refers to a direction away from the head toward the tail of the patient. A “cranial view” of an object is a view from a perspective looking at the cranial surface or aspect of the object. A “caudal view” is a view from a perspective looking at the caudal surface or aspect of the object. The term “medial” refers to a direction toward the center of the patient's body mass and the term “lateral” refers to a direction away from the center of the patient's body mass. The term “dorsal” refers to a direction toward the patient's spine and the term “ventral” refers to a direction away from the patient's spine. For the purposes of this application, the apparatus and method are described using these terms in the context of a veterinary patient. It is understood that in the context of a human patient, the cranial/caudal and dorsal/ventral directions will differ.

As used herein, the term “proximal” refers to a direction toward the point of origin or attachment of a limb. As used herein, the term “distal” refers to a direction away from the point of origin or attachment of a limb. The terms “longitudinal” and “axial” refer to an axis extending in the proximal and distal directions, unless otherwise expressly defined.

In some examples, values, procedures, or apparatus may be referred to as “lowest,” “best,” “minimum,” or the like. It will be appreciated that such descriptions are intended to indicate that a selection among many alternatives can be made, and such selections need not be better, smaller, or otherwise preferable to other selections.

In the following description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

Unless otherwise indicated, all numbers expressing dimensions (e.g., heights, widths, lengths, etc.), angles, quantities of components, percentages, temperatures, forces, times, and so forth, as used in the specification or claims, are to be understood as being modified by the term “about.” Accordingly, unless otherwise indicated, implicitly or explicitly, the numerical parameters set forth are approximations that can depend on the desired properties sought and/or limits of detection under test conditions/methods familiar to those of ordinary skill in the art. When directly and explicitly distinguishing embodiments from discussed prior art, the embodiment numbers are not approximates unless the word “about” is recited. Furthermore, not all alternatives recited herein are equivalents.

### Exemplary Embodiments

Disclosed herein are embodiments of implantable orthopedic devices and assemblies that can be used to correct biomechanical misalignment of a long bone in multiple planes in both human and veterinary applications. Thus, although the examples herein pertain primarily to veterinary orthopedic procedures, it should be understood that the devices and methods described herein are also applicable to orthopedic procedures in humans For example, the orthopedic devices described herein can be used to help restore and/or replace functionality as part of various procedures including sliding humeral osteotomies (SHO) to correct the loading of the elbow joint, in triple pelvic osteotomies (TPO) to correct hip dysplasia, as well as in femoral neck angle corrections, femoral version corrections, tibial plateau leveling osteotomy (TPLO) procedures, and patellar luxation corrections (e.g., through tibial tuberosity transposition).

In human and animal orthopedics, straight osteotomies, radial osteotomies, and spherical osteotomies can be created at specific locations in long bones to achieve realignment of a bone segment to the overall limb axis for improved biomechanics and/or to help restore and/or replace the functionality of defective ligaments. There are multiple physiological problems associated with long bones that can affect limb biomechanics, which can occur as a result of trauma (e.g., bone fractures that heal in a misaligned position), and/or birth defects. Surgical methods of re-establishing appropriate biomechanics of a limb can include repositioning proximal and distal bone segments to correct alignment issues. There are clinical examples for many long bones (e.g., femur, tibia, humerus, radius, ulna, etc.), which can be managed through corrective osteotomies to restore improved limb function. With reference to the tibia, there are proximal and distal corrective osteotomies that can address different biomechanical alignment issues.

### Example 1

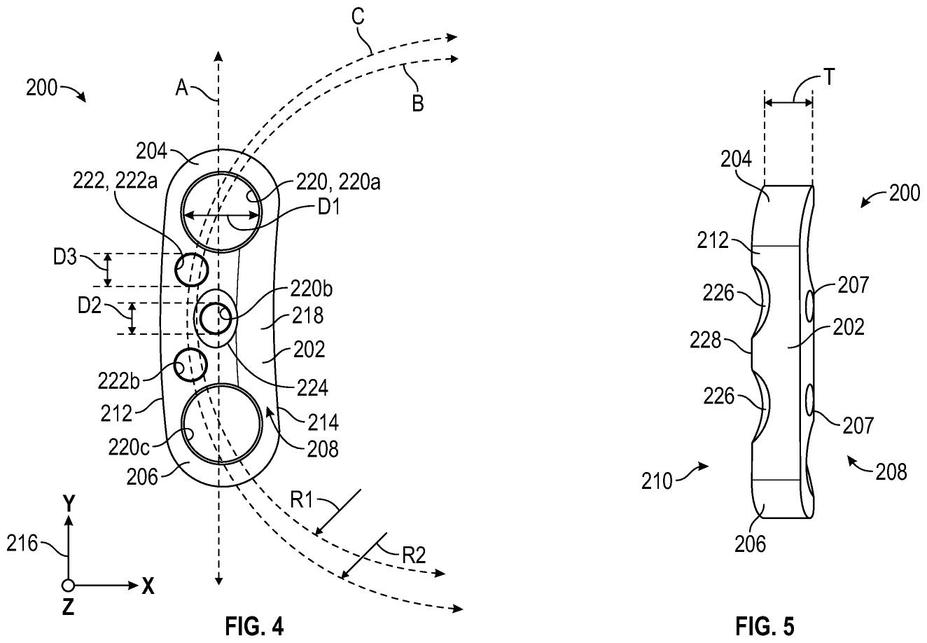

For example, in certain embodiments, the orthopedic implants described herein can include a spacer couplable to one or more orthopedic implants (e.g., bone plates, etc.). The spacer can be configured to be coupled to any of various implants intended for various clinical procedures in order to provide biomechanical alignment in one or more planes similar to a stepped plate. For example, in a TPLO procedure, a spacer (e.g., spacer 200 shown in FIGS. 4-5) can be coupled to a bone plate 100, such as the BioMedtrix TPLO Curve® bone plate, to provide additional biomechanical alignment by medializing the osteotomy to realign the tracking of the patella. Though the bone plate 100 and spacer 200 are described herein with reference to a TPLO procedure, it should be noted that the bone plate 100 and spacer 200 can be used in any of various procedures on various other long bones, and that the spacer 200 can be used with any of various other orthopedic implants.

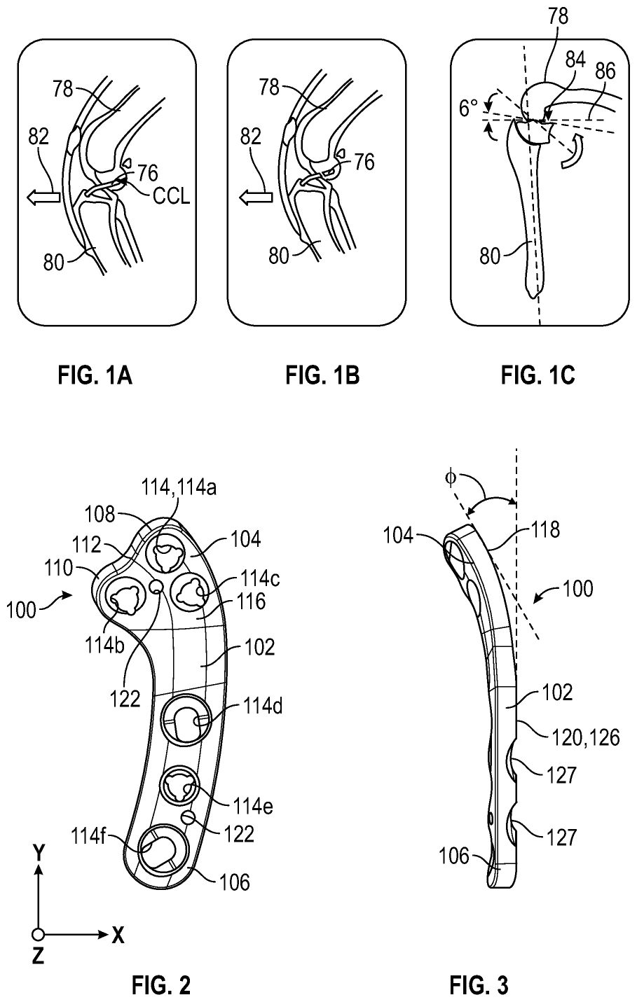

In veterinary medicine, a tibial plateau leveling osteotomy (TPLO) can be performed to re-position the tibial plateau to, for example, function as a buttress to resist certain physiological movements or address rupture of the anterior (cranial) cruciate ligament. A TPLO may be performed to compensate for ruptures of the cranial cruciate ligament (for example, in dogs). A representative example of a TPLO to repair a ruptured cranial cruciate ligament is illustrated in FIGS. 1A-1B. As illustrated in FIG. 1A, the cranial cruciate ligament 76 can extend between the femur 78 and the tibia 80, and can resist advancement of the tibia in the direction indicated by arrow 82 due to force applied to the tibia by the femur. FIG. 1B illustrates forward advancement of the tibia 80 in the direction of arrow 82 due to rupture of the cranial cruciate ligament 76. In a typical example, an osteotomy can be made in the lateral plane on the medial side of the tibia 80 to reduce the angle θ of the tibial plateau 84 with respect to a horizontal reference plane 86, as illustrated in FIG. 1C. This can create a mechanical abutment in the caudal aspect of the knee, assisting the soft tissues in preventing the femur from sliding off the back of the tibia, mitigating the effects of a non-functional cranial cruciate ligament.

Bone plates used in association with osteotomy procedures such as the procedures described herein can provide one or more functions, for example, stabilization of the osteotomy and compression of the osteotomy. In some embodiments, the bone plates described herein can incorporate one or more compression slots or compression anchor openings that utilize an internal ramp within the side walls of the anchor opening to apply compression to the underlying osteotomy.

FIGS. 2-3 illustrate an exemplary bone plate 100. The bone plate 100 can have an elongated body 102 including a first end portion 104 (also referred to as a proximal end portion) and a second end portion 106 (also referred to as a distal end portion). With reference to the coordinate axes of FIG. 2, the bone plate 100 can be curved in the x-y plane such that the first end portion 104 is offset from the second end portion 106 in a direction along the x-axis. The first end portion 104 can have a generally curved shape, and can include a proximal lobe 108 and a distal lobe 110 separated from one another by a recessed portion 112. The first end portion 104 can also define one or more openings 114 extending through a thickness of the bone plate 100 and configured as anchor openings. For example, in the illustrated embodiment the first end portion includes three openings 114a, 114b, 114c for receiving any of a variety of bone fixation anchors, such as screws. The openings 114 can be arranged in a generally triangular arrangement, with a first opening 114a being located on the proximal lobe 108, a second opening 114b being located on the distal lobe 110, and a third opening 114c being intermediate the first and second openings 114a, 144b and offset from the openings 114a, 114b along the x-axis. In some embodiments, the location of the anchor openings can be associated with optimal bone cross sections (for example, areas in which the cortical bone is thicker to aid in fixation), although it should be appreciated that the first end portion can include any suitable number of anchor openings located at any suitable location.

Referring still to FIG. 2, the second end portion 106 can have a generally curved shape, and can have a width dimension that is less than a width dimension of the first end portion 104. For example, in the illustrated embodiment, the first and second end portions can be joined by a transition region generally indicated at 116, in which the width of the bone plate tapers from the relatively wider first end portion 104 to the relatively narrower second end portion 106.

The second end portion can also define one or more openings 114 extending through the thickness of the bone plate 100 and configured as anchor openings. For example, in the illustrated embodiment the second end portion 106 can define three anchor openings 114d, 114e, 114f, with anchor openings 114d and 114f being configured as compression anchor openings, although the second end portion can include any suitable number of anchor openings in any suitable configuration. In some embodiments, compression anchor opening 114d can be aligned with anchor opening 114a, which can be generally representative of the direction of movement of the bone plate 100 when an anchor (e.g., a screw) is inserted through the compression anchor opening 114d, can provide increased compression between bone segments at the osteotomy site while reducing movement of the bone segments relative to one another.

In some embodiments, the bone plate 100 can have an outer plate contour (e.g., medial or lateral) such that overhang of a long bone to which the bone plate is affixed is reduced or prevented. Reducing overhang can be important to avoid soft tissue impingement, which can result in reduced range of motion and pain post-operatively. The bone plate can also be made in left or right configurations to accommodate long bones on different sides of the body. For example, the bone plate illustrated in FIGS. 2-3 is a left-handed configuration.

In some embodiments, the bone plate 100 can be curved in multiple planes to maintain a geometry capable of placement within the boundaries of a bone following an osteotomy. For example, in addition to the curvature of the bone plate 100 in the x-y plane as shown in FIG. 2, the bone plate can also be curved along the z-axis out of the x-y plane, as shown in FIG. 3. More specifically, in the illustrated embodiment, the first end portion 104 can be angled in the z-direction to allow the first end portion to conform to the shape of the proximal or distal portion of a bone to which the bone plate is applied. In some embodiments, a plane defined by the lower surface 118 of the first end portion 104 can define an angle ϕ with a plane defined by the lower surface 120 of the second end portion 106 of from 5 degrees to 40 degrees. In some embodiments, the angle ϕ can be from 15 degrees to 40 degrees. In some embodiments, the angle ϕ can be 25 degrees. The angle ϕ may be varied from application to application, including by being bent to a desired angle by a surgeon during an osteotomy procedure, as required. Further details of the bone plate 100 can be found, for example, in U.S. Pat. No. 10,226,288, which is incorporated by reference herein in its entirety.

The bone plate 100 can further comprise one or more pin openings 122 extending through a thickness of the bone plate 100. During implantation of the bone plate 100, one or more fasteners can extend through the pin openings 122 to initially stabilize the bone plate to the subject's bone.

In some embodiments, the bone plate 100 can be made of any biocompatible metal such as, for example, stainless steel, titanium, etc. In some embodiments, the bone plate 100 can comprise any of various biocompatible polymers or plastics, including polylactic acid, or other aliphatic polymers. When fabricated from polylactic acid, for example, the bone plate can be configured to be naturally resorbed or dissolved by the body after a period of time has elapsed sufficient to allow the osteotomy to heal. For example, in some embodiments the bone plate can be configured to dissolve over a period of from 8 weeks to 12 weeks.

FIGS. 4-5 illustrate an exemplary embodiment of a spacer 200, according to one embodiment. The spacer 200 can be coupled to a bone plate, such as bone plate 100 described previously, to shift the osteotomy medially or laterally and realign patella tracking and/or eliminate luxation.

Referring to FIG. 4, the spacer 200 can be configured as a member including a main body 202 comprising a first end portion 204, a second end portion 206, a first surface 208, a second surface 210 (FIG. 5), a first edge 212, and a second edge 214. In the illustrated embodiment, the main body 202 can have an elongated square-oval shape wherein the first and second end portions 204, 206 are rounded. In other embodiments, the main body 202 can have any of various shapes. For example, in some embodiments, the end portions 204, 206 can be square, triangular, or chamfered in lieu of or in addition to being rounded.

As shown in FIG. 4, the spacer 200 can have an overall curved shape. With reference to coordinate axes 216, the spacer 200 can be curved in the x-y plane such that the first end portion 204 and the second end portion 206 are offset from a center portion 218 in a direction along the x-axis. In other words, the first and second end portions can curve relative to a longitudinal axis A of the spacer 200. In the illustrated embodiment, the spacer 200 is curved such that the first edge 212 is convex and longer than the second edge 214, which is concave. In some embodiments, the edges 212 and 214 can have the same radii, and in other embodiments, the first and second edges can have different radii. However, in other embodiments, the spacer 200 can curve in the opposite direction. In certain examples, the direction of the curve can be selected depending on the particular procedure to be performed, the species of the patient, the size of the patient, the selected implantation site, etc.

In certain embodiments, the degree of curvature of the spacer 200 can be defined as the reciprocal of the radius of a circle comprising the second edge 214 (and/or the first edge 212) as an arc, as shown in the following equation:

$$\\ \begin{matrix}{K}_{s} = \frac{1}{R} ; & Equation 1\end{matrix}$$

where Kₛ=the curvature of the spacer, and R=the radius of a circle comprising the second edge 214 as an arc of the circle. The degree of curvature of the spacer 200 can be selected to match the degree of curvature of an existing orthopedic implant (e.g., bone plate 100) to which the spacer 200 can be coupled.

The spacer 200 can comprise a variety of openings 220 extending through a thickness of the main body 202. In the illustrated embodiment, the spacer 200 comprises a subset of three openings 220a, 220b, and 220c. Openings 220a and 220c can be referred to as “first openings” and together form a set of first openings, and opening 220b can be referred to as an “anchor opening”. In the illustrated embodiment, first opening 220a is defined in the first end portion 204 and first opening 220c is defined in the second end portion 206. The anchor opening 220b can be defined in the center portion 218. The first openings 220a, 220c can have a diameter D₁ greater than a diameter D₂ of the anchor opening 220b. In certain embodiments, the diameter D1 can be configured to allow the spacer member 200 to move/travel with the bone plate 100 during tightening of bone screws in the compression slots 114d and/or 114f. Such motion can also be relative to bone screws inserted through the compression slots 114d and/or 114f of the bone plate. In other embodiments the openings 220a and/or 220c need not be circular, but can be configured as slots or tracks having lengths sufficient to allow the spacer member to move relative to bone screws inserted through the compression slots in the bone plate.

The spacer 200 can further comprise one or more pin openings 222 extending through a thickness of the spacer. In the illustrated embodiment, the spacer 200 can comprise first and second pin openings 222a, 222b. Openings 222a and 222b can be referred to as “second openings” and together form a set of second openings. The first pin opening 222a can be disposed adjacent the first end portion 204 of the main body 202, and the second pin opening 222b can be disposed adjacent the second end portion 206. Each pin opening 222 can have a diameter D₃ less than the diameter D₁ of the first openings 220.

In some embodiments, such as the illustrated embodiment, both pin openings 222 can be disposed on the same side of the main body relative to the longitudinal axis A extending through a length of the spacer 200. Such a configuration can allow the same spacer 200 to be used with either a right-handed or a left-handed bone plate 100 by rotating the spacer 200 about an axis extending into and out of the page through anchor opening 220b such that either the first or second pin opening 222a, 222b aligns with the pin opening 122 of the bone plate 100.

In some embodiments, one or more openings can include a recessed portion 224 in the first surface 208 surrounding the opening. For example, in the illustrated embodiment, anchor opening 220b is disposed within a recessed portion 224.

The openings 220, 222 of the spacer 200 can be configured to align with the openings of the second end portion 106 of the bone plate 100 (e.g., openings 114d, 114e, 114f, and 122), such that fasteners or anchors (e.g., screws, pins, etc.) extending through the openings in the bone plate 100 also extend through the openings 220, 222 in the spacer 200. The first openings 220a, 220c can be arranged on a curve such that a circle B extending through a center point of each first opening 220a, 220c has a radius R₁. The pin openings 222 can be arranged on a curve such that a circle C extending through a center point of each pin opening 222a, 222b has a radius R₂. The radius R₂ can be different than the radius R₁. For example, R₂ can be greater than R₁.

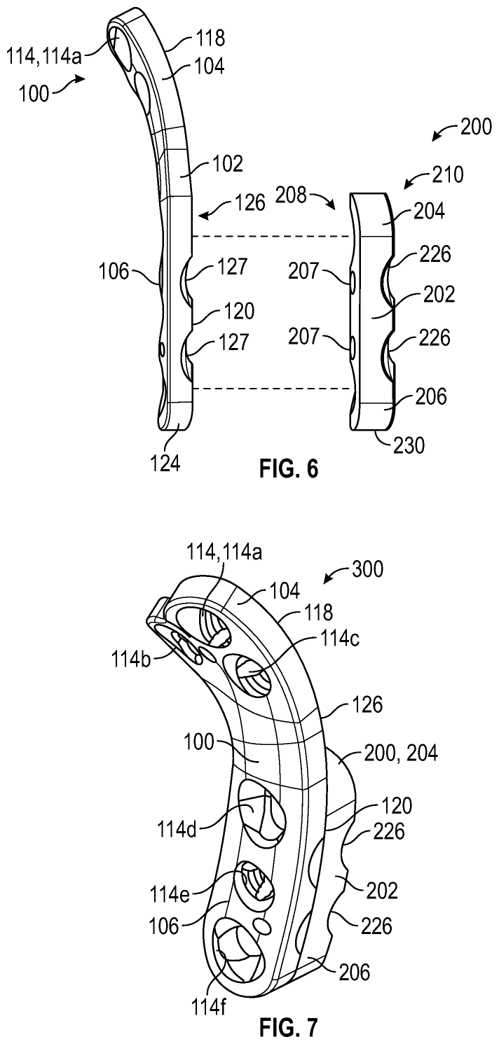

Referring to FIG. 5, the first surface 208 of the spacer 200 can comprise one or more convex protrusions 207. As shown in FIG. 6, the protrusions 207 can be arranged such that when the spacer 200 is coupled to a bone plate 100, the protrusions 207 mate with corresponding recesses/cutouts 127 in a first surface 126 of the bone plate 100. The mating of the protrusions 207 and recesses 127 can help prevent or mitigate movement of the spacer 200 relative to the bone plate 100 when the components are coupled together.

Referring again to FIG. 5, the second surface 210 of the spacer 200 can comprise one or more recesses/cutouts 226 defining a scalloped surface. The scalloped surface can be configured to reduce contact between the spacer and the surface of the underlying bone. The surface portions 228 between and on either side of the cutouts 226 can be the primary points of contact between the spacer 200 and the bone surface, thereby preventing or reducing damage to the periosteum. This can promote improved healing of the bone since the periosteum contains fibroblasts and progenitor cells that develop osteoblasts for maintaining and healing bone. In the illustrated embodiment, the spacer 200 includes two cutouts 226, however, in other embodiments, the spacer 200 can include any number of cutouts having any size, including no cutouts, as desired.

In some embodiments, the overall shape of the spacer 200 can be curved in multiple planes to maintain a geometry capable of placement within the boundaries of a bone following an osteotomy. For example, in addition to the curvature of the spacer 200 in the x-y plane, as mentioned previously and as shown in FIG. 4, in some embodiments, the spacer 200 can also be curved along the z-axis out of the x-y plane such that the spacer has a dished configuration. In such embodiments, the curvature of the spacer 200 in the z-direction can be configured to match the curvature of a corresponding bone plate, e.g., bone plate 100.

In some embodiments, the spacer 200 can be made of any biocompatible material such as, for example, biocompatible metal (e.g., stainless steel, titanium, cobalt-chromium, and/or tantalum, etc.), biocompatible polymer (e.g., polyether ether ketone (PEEK)), and combinations thereof. In some embodiments, the spacer 200 can comprise any of various biocompatible and bioresorbable materials. For example, the spacer can be configured to be naturally resorbed or dissolved by the body after a period of time has elapsed sufficient to allow the osteotomy to heal. In still other embodiments, the spacer can comprise a combination of non-resorbable biomaterials and bioresorbable materials such that only portions of the spacer can be dissolved.

As shown in FIGS. 6-7, the spacer 200 can be coupled to a bone plate, such as TPLO bone plate 100, to form a spacer assembly 300. As shown in FIG. 6, a lower (e.g., distal) edge 230 of the spacer 230 can be aligned with a lower (e.g., distal) edge 124 of the bone plate 100 such that the spacer 200 aligns with the second end portion 106 of the bone plate 100. The spacer 200 can be disposed on the surface 126 of the second end portion 106 that is configured to be positioned adjacent the bone, such that the second surface 210 of the spacer 200 contacts the bone in lieu of the second end portion 106 of the bone plate 100. In certain embodiments, the length of the spacer member can be less than the length of the bone plate such that the combined thicknesses of the second end portion 106 of the bone plate and the spacer member result in an increased thickness portion which can be configured to achieve a specified offset between bone segments.

The spacer 200 can be coupled to the bone plate 100 during an implantation procedure using fasteners or anchors (e.g., screws, pins, etc.) that extend through the openings in the bone plate 100, through the spacer 200, and into the native bone. In other embodiments, the spacer 200 and bone plate 100 can be coupled together using, for example, welding, adhesives, or mechanical fasteners apart from those that extend fully through the assembly.

The openings 114 in the bone plate 100 and/or the openings 220 in the spacer 200 can be configured for use with locking screws, non-locking screws, or combinations thereof, which can be driven into a bone to secure the spacer assembly 300 to the bone. In some embodiments, the spacer assembly 300 can be used in combination with one or more locking bone screws, which can provide a locking feature in the head of the screw that locks or engages the screw with the bone plate 100 and/or spacer 200 when the screw is inserted into the bone. In some embodiments, using locking screws can help prevent the spacer assembly 300 from being compressed against the bone and damaging the periosteum. Certain embodiments of locking screws that may be used in combination with the bone plates described herein are disclosed in U.S. Pat. No. 8,696,715, which is incorporated herein by reference in its entirety.

The spacer 200 can have a selected thickness T (FIG. 5) such that when the spacer assembly 300 is implanted at an osteotomy the selected offset between the upper and lower portions of the bone is created to provide biomechanical alignment similarly to a stepped plate. In some embodiments, the thickness T of the spacer can be from 1 mm to 10 mm. In some embodiments, the thickness T can be from 1 mm to 5 mm. In some embodiments, the thickness T can be about from 3 mm to 6 mm.

It should be understood that the bone plates, spacers, and methods described herein are applicable to any long bones in canids, as well as in other mammals including felines and humans. More specifically, the ability to adjust the biomechanical alignment of a bone along multiple axes can allow the spacer assembly 300 to be used with a variety of long bones and a variety of osteotomy procedures and/or fractures of those bones. The spacer assemblies described herein can also be used on multiple sides of the body, and at the proximal and distal ends of bones without significant modification, contrary to known bone plates.

In certain embodiments, the spacer member 200 can be shaped and configured for coupling to the first end portion 104 of the bone plate.

The spacer assemblies described herein can provide improved initial stability, improved compression at the osteotomy interface, and improved biomechanical alignment which can yield faster healing in the desired realigned position. In some embodiments, this can be accomplished with a single bone plate family (including multiple sizes) in left and right orientations, and spacers in various sizes (e.g., thicknesses, lengths, etc.) as opposed to multiple families of bone plates and stepped plates with multiple iterations in each family.

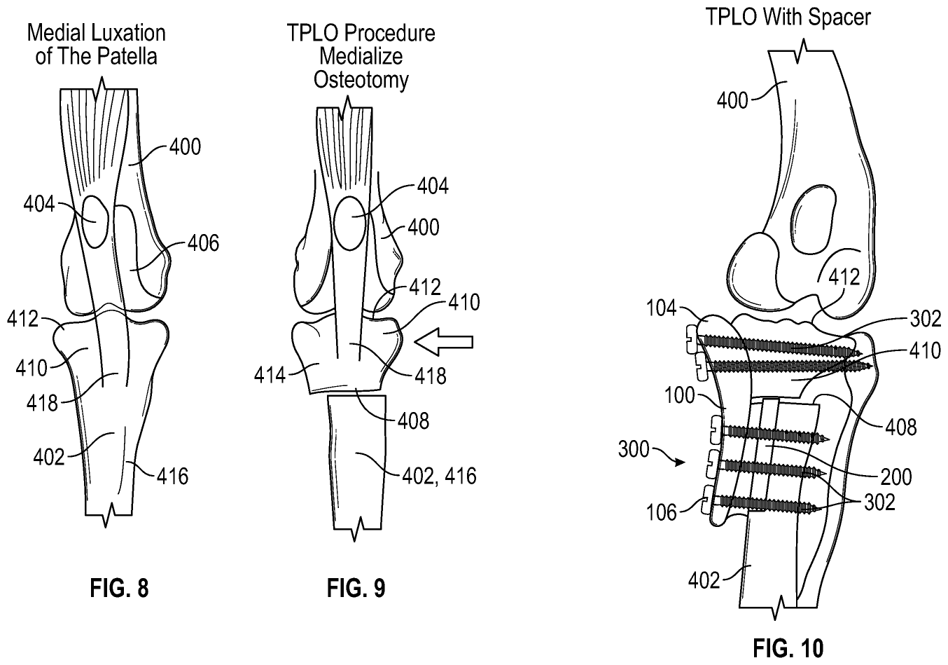

The disclosed spacer assembly embodiments can be implanted as part of a TPLO procedure as described in the following exemplary method. Referring to FIG. 8, a ruptured cranial cruciate ligament (CCL) connecting the femur 400 and the tibia 402 can cause the patella 404 to luxate out of the femoral trochlear groove 406, as shown in FIG. 8. Accordingly, during a TPLO procedure it can be advantageous to shift the osteotomy medially using a spacer assembly, such as spacer assembly 300, to realign the patella 400 and mitigate or eliminate the luxation issue.

For example, as shown in FIG. 9, a surgeon can cut an osteotomy 408 through the head 410 of the tibia 402 such that the tibial plateau 412 and a portion of the tubercle 414 are separated from the main body/shaft 416 of the tibia 402, forming a proximal portion (e.g., the head portion 410 including the tibial plateau 412 and a portion of the tubercle 414) and a distal portion (e.g., the main body/shaft 416 of the tibia). The osteotomy 408 can be positioned below (e.g., distal to) the site of the patellar ligament attachment 418. The osteotomy 408 can be a substantially straight cut located distal to the native insertion sites of the caudal cruciate ligament and the medial collateral ligament. This location can mitigate the risk of soft tissue damage such as, for example, cutting the caudal cruciate and medial collateral ligaments. Furthermore, the osteotomy location can mitigate surgical trauma to the patient, thus minimizing recovery time, because the joint capsule does not need to be incised.

Referring to FIG. 10, once the cut has been made, the surgeon can couple the spacer assembly 300 to the tibia 402 to medialize the osteotomy 408. That is, to shift the proximal portion (e.g., the head 410) and the distal portion (e.g., the shaft 416) relative to one another such that a longitudinal axis of the proximal portion is offset from a longitudinal axis of the distal portion. For example, the first end portion 104 of the bone plate 100 can be coupled to the head portion 410 of the tibia 402 and the second end portion 106 can be located generally over the portion of the tibia distal to the osteotomy 408. The spacer 200 can be coupled to the bone plate 100 such that it is positioned distal to the osteotomy 408, as shown, and such that it supports the medialized osteotomy (e.g., such that it supports the portion of the proximal portion that overhangs the distal portion).

The spacer assembly 300 can be initially stabilized to the bone via fasteners extending through pin openings 122 (FIG. 2), 222 (FIG. 4).

Once the spacer assembly 300 is positioned on the bone, it can be anchored to the bone using one or more bone screws 302 extending through the one or more openings 114, 220 in the bone plate 100 and spacer 200, respectively. In the illustrated embodiment, two bone screws 302 are used to couple the first end portion 104 of the bone plate 100 to the head portion 410 of the tibia 402, and three bone screws 302 are used to coupled the second end portion 106 and the spacer 200 to the portion of the tibia 402 distal to the osteotomy 408. In other embodiments, the spacer assembly 300 can comprise a greater or lesser number of openings 114, 220 and can be anchored to the bone using a correspondingly greater or lesser number of bone screws 302.

In some embodiments, the bone screws 302 can be anchored to the bone through the openings 114, 220 in a particular order. The sequence of use of these openings can be specific to surgical techniques for particular long bone osteotomies. In some embodiments, the objective can be to establish initial fixation between the plate and the bone, then to provide compression to the osteotomy site, and then secure the bone plate and bone together. Each anchor opening and compression anchor opening can be marked with a number indicating its position in the sequence of anchor insertion for a particular operation. Further details of the anchoring process can be found, for example, in U.S. Pat. No. 10,226,288.

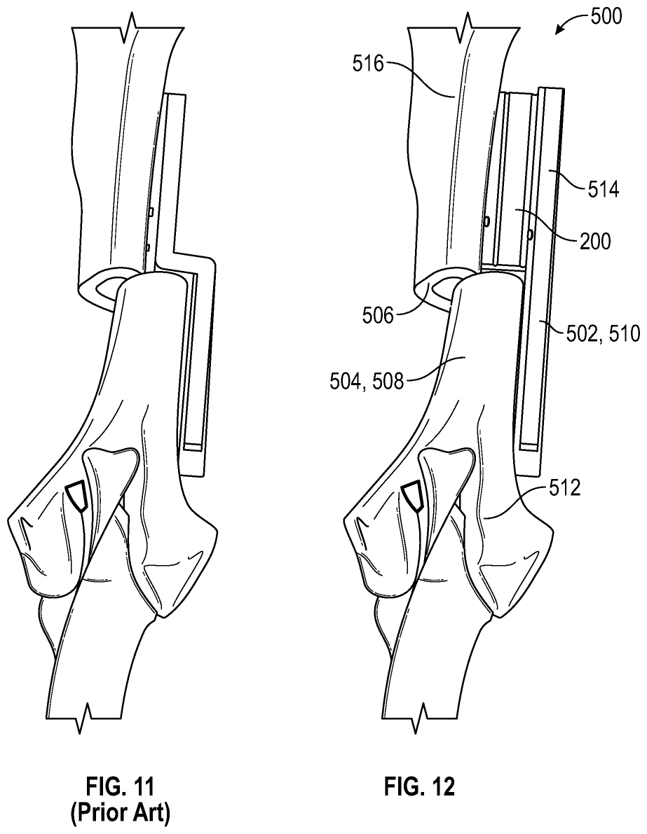

In other embodiments, a spacer assembly can be implanted as part of a sliding humeral osteotomy (SHO). A SHO procedure can be used to correct medial compartment disease in a subject by shifting a portion of the humerus medially. For example, FIG. 11 illustrates a prior art stepped plate assembly used in a SHO procedure.

Referring to FIG. 12, a spacer assembly 500 can include a spacer 200 according to another embodiment, and a bone plate 502. In certain embodiments, the bone plate 502 can have a symmetrical, non-curved design. For example, the bone plate 502 can be a BioMedtrix MultiPurpose Bone Plate®. In certain examples, the spacer member 200 can comprise a straight, non-curved main body including a plurality of openings, at least a portion of which can be aligned with openings of the bone plate 502 when the spacer and the bone plate are assembled together. A spacer assembly 500 can be implanted as part of a SHO procedure as described in the following exemplary method. Elbow dysplasia (e.g., medial compartment disease (MCD)) can result from severe erosion of cartilage on the medial aspect of the humerus 504. SHO procedures can realign the limb in order to mitigate or eliminate the application of force on areas having damages or reduced cartilage and shift the application of force to areas having undamaged cartilage.

For example, as shown in FIG. 12, a surgeon can cut a mid-humeral osteotomy 506, and can slide the distal segment 508 of the humerus 504 in a medial direction. The surgeon can then couple the spacer assembly 500 to the humerus. For example, the first end portion 510 of the bone plate 502 can be coupled to the portion of the humerus adjacent the elbow joint 512, and the second end portion 514 can be located generally over the body 516 of the humerus 504 such that the spacer 200 is positioned proximal to the osteotomy 506. The spacer assembly 500 can be initially stabilized to the bone via fasteners and then can be anchored to the humerus 504 using bone anchors, such as bone screws 302 shown in FIG. 10.

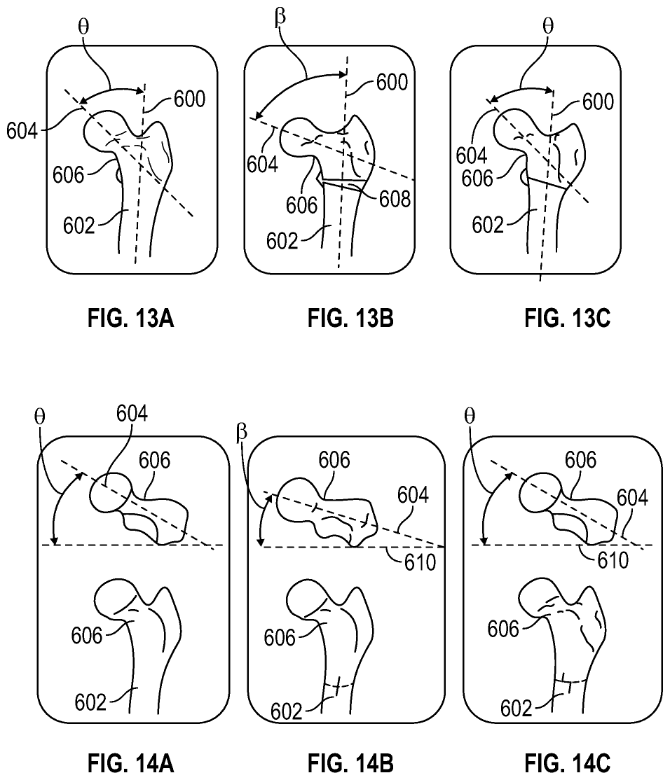

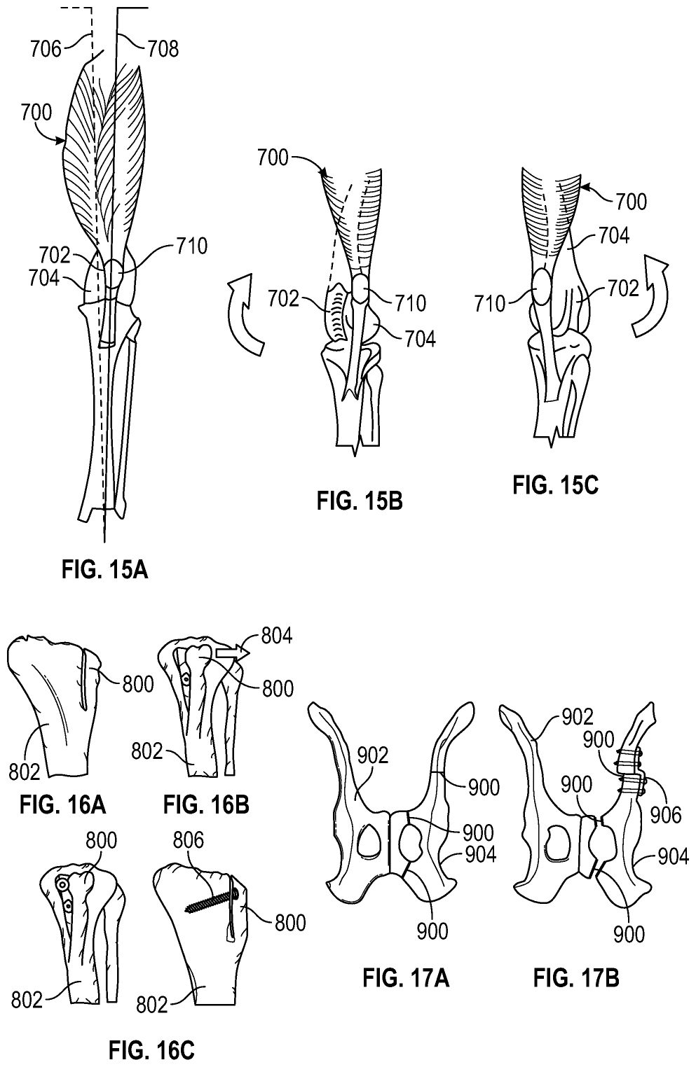

Spacer embodiments such as those described herein can be used in a variety of osteotomy procedures that require adjustments in more than one plane. For example, spacers can be used in femoral neck angle correction (FIGS. 13A-13C), femoral version correction (FIGS. 14A-14C), to correct patellar luxations (FIGS. 15A-15C) such as by using a TPLO procedure as described previously, and in tubercle transposition procedures (FIGS. 16A-16C).

For example, a representative femoral neck angle correction is shown in FIGS. 13A-13C. FIG. 13A illustrates a normal angle θ between a longitudinal axis 600 of a femur 602 and a longitudinal axis 604 of the femoral neck 606. FIG. 13B illustrates a case in which the angle β between the longitudinal axis 600 of the femur 602 and the longitudinal axis 604 of the femoral neck 606 is greater than normal. The angle of the femoral neck can be corrected by performing an osteotomy (e.g., with flat saw blade) to remove a wedge-shaped portion 608 of the femur (FIG. 13B) to locate the femoral neck at the desired angle θ (FIG. 13C).

A representative femoral version correction is shown in FIGS. 14A-14C, wherein the femoral anteversion angle θ between the longitudinal axis 604 of the femoral neck 606 is corrected relative to a horizontal plane 610. FIG. 14A illustrates a normal femoral anteversion angle θ, while FIG. 14B illustrates a femoral anteversion angle β that is less than normal. By performing an osteotomy (e.g., with a flat saw blade) to rotate the femoral neck 606, the anteversion angle can be corrected, as shown in FIG. 14C.

Patellar luxation is another example of a pathology that may be addressed by osteotomy procedures. With reference to FIGS. 15A-15C, patellar luxation can occur when there is a misalignment between the quadriceps mechanism generally indicated at 700 and the trochlear groove 702 of the distal femur 704, and/or when there is a disparity between the loading axis 706 of the leg and the anatomical axis 708, as illustrated in FIG. 15A. In such circumstances, when the knee is flexed, the patella 710 can travel out of the trochlear groove 702, or luxate, resulting in pain and limited motion and function. Lateral luxation of the patella 710 is illustrated in FIG. 15B, while medial luxation is illustrated in FIG. 15C.

Tubercle transposition is another example of a procedure used to address patellar luxation. With reference to FIGS. 16A-16C, a tubercle transposition can involve cutting the tubercle 800 of the tibia 802, as shown in FIG. 16A. The cut tubercle 800 can then be shifted laterally, as shown by arrow 804 in FIG. 16B. Once shifted, the position of the tubercle 800 can be stabilized using a bone anchor 806, as shown in FIG. 16C. Tubercle transposition is often combined with a TPLO procedure to address the cranial cruciate ligament. By creating a TPLO osteotomy and shifting the osteotomy using a spacer assembly (e.g., spacer assembly 300), the patella can be realigned, preventing luxation. Using a spacer assembly can minimize morbidity and allow the surgeon to perform the TPLO procedure using a spacer assembly to address the patellar alignment correction concurrently. Accordingly, the spacer assembly embodiments described herein can allow multiple orthopedic issues to be solved using one procedure by producing realignment in two planes.

Hip dysplasia is another pathology that may be addressed by osteotomy procedure. With reference to FIGS. 17A-17B, hip dysplasia occurs when there is increased laxity of the hip joint. Osteotomies 900 are performed in the pelvis 902 (e.g., at the pubis, the ischium, and the shaft of the ilium) such that the pelvic socket or acetabulum 904 can be rotated relative to the femoral head to force the femoral head more deeply into the acetabulum 904 when the subject walks. The osteotomies can then be stabilized using a stepped bone plate 906, as shown in FIG. 17B, or alternatively, a spacer assembly such as the spacer assemblies described previously.

### Example 2

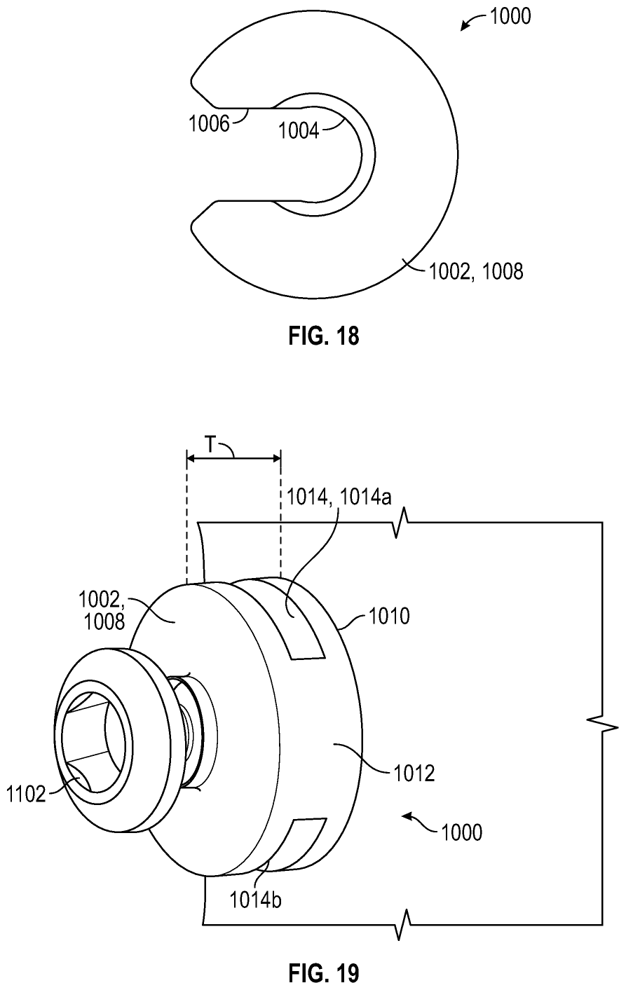

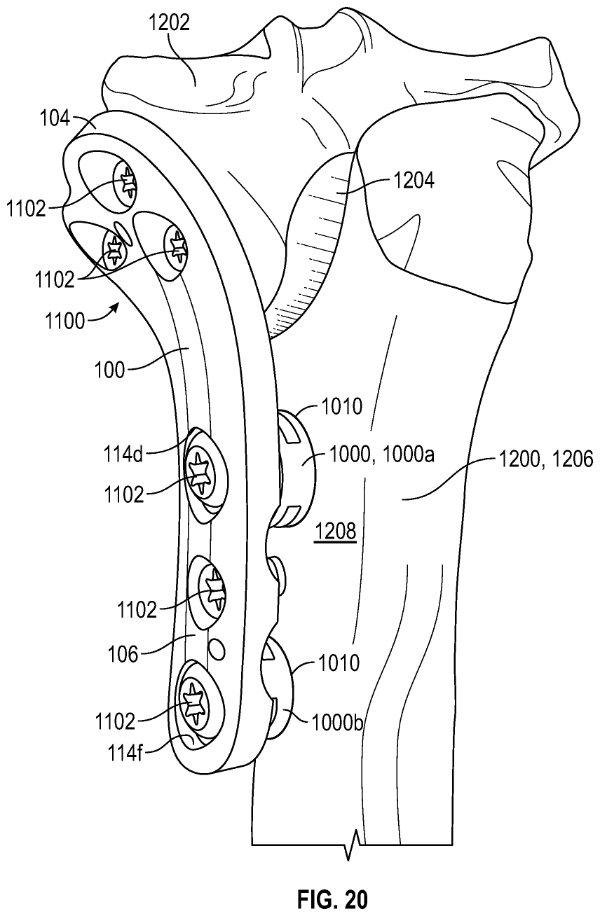

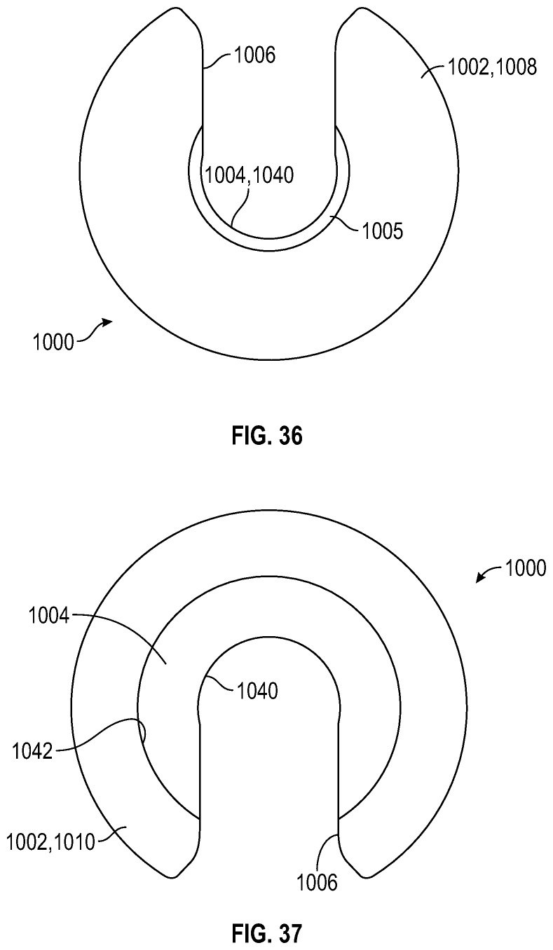

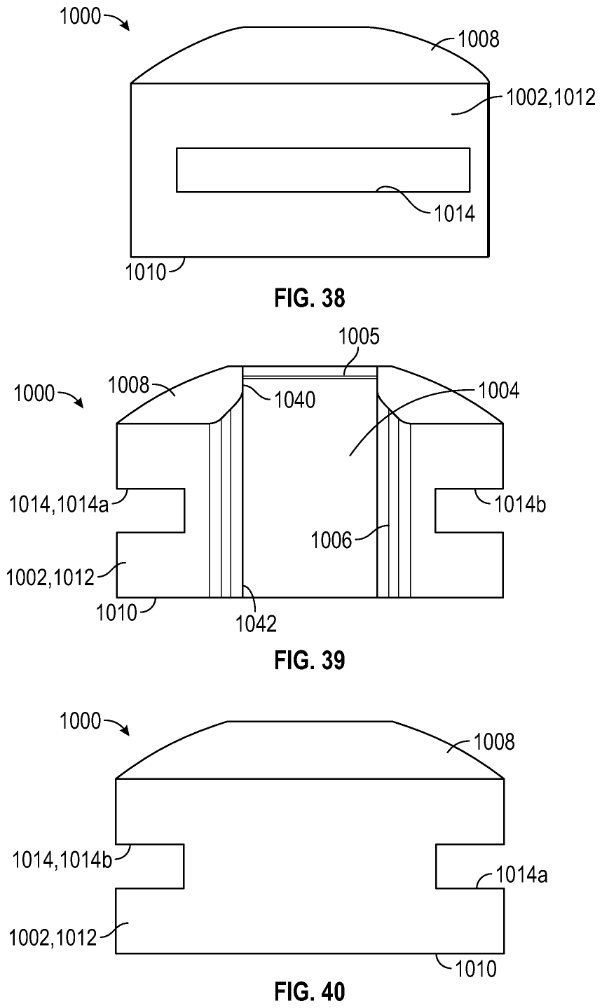

FIGS. 18-19 and 34-40 illustrate another exemplary embodiment of a spacer 1000. In some embodiments, in lieu of or in addition to spacer 200, one or more spacers 1000 can be used with a bone plate, for example bone plate 100, to form a spacer assembly 1100 (FIG. 20). As described previously with respect to spacer 200 and spacer assembly 300, the spacer 1000 and spacer assembly 1100 can be used to shift or realign bone portions at an osteotomy medially or laterally, realign patella tracking, and/or eliminate luxation.

Referring to FIGS. 18-19 and 34-40, each spacer 1000 can have a main body 1002 with an overall C-shape, including an inner bore or central aperture 1004 extending in a longitudinal direction through a thickness T (FIG. 19) of the main body, and a cutout or opening 1006 (also be referred to as a slot) that also extends through the thickness T. The slot 1006 can be configured (e.g., sized and shaped) to receive a fastener such that the spacer can be disposed around a fastener 1102 (FIG. 19) (e.g., around the shaft of a screw) by sliding the fastener through the slot 1006. For example, as shown in FIG. 18, the slot 1006 can extend from the central aperture 1004 to a radially outer edge of the spacer 1000. Referring to FIG. 19, the spacer 1000 can have a first surface 1008, a second surface 1010 opposite the first surface, and a sidewall 1012 extending between the first and second surfaces 1008, 1010. In the illustrated embodiment the outer perimeter of the main body 1002 is circular, although the main body can have other non-circular shapes such as flat or planar edge portions, etc.

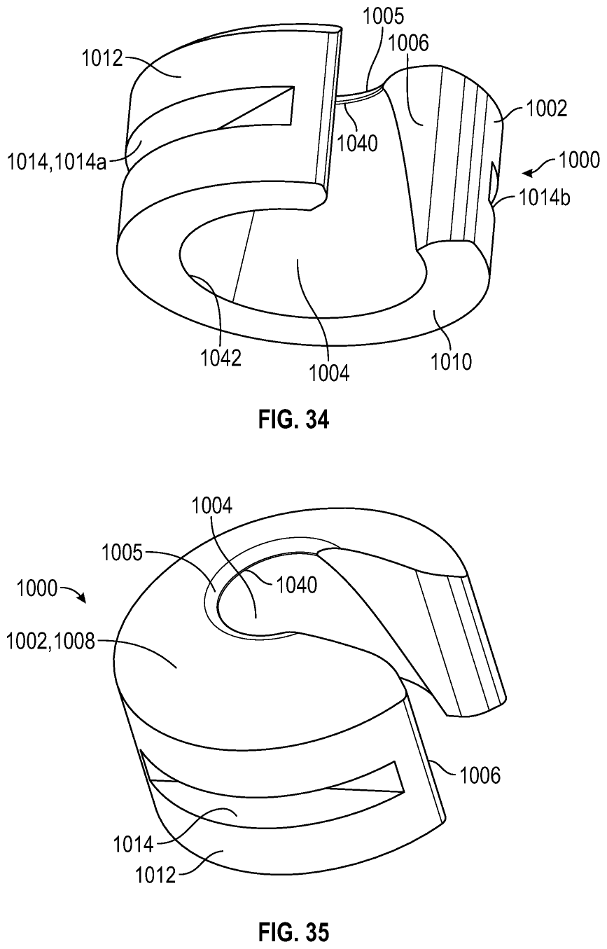

Referring still to FIG. 19, the first and/or second surfaces 1008, 1010 can be curved surfaces having a convex, domed shape. For example, in the illustrated embodiment, the first surface 1008 has a convex shape and the second surface 1010 has a flat shape (see e.g., FIG. 34). In some embodiments, the first surface 1008 can be shaped such that when the spacer 1000 is disposed adjacent the bone plate 100, the convex or domed shape of the surface 1008 mates with a curved surface (e.g., the first surface 126) of the bone plate 100. The mating of the surface 1008 and the curved surface of the bone plate 100 can help prevent or mitigate movement of the spacer 1000 relative to the bone plate 100 when the components are coupled together.

The smaller surface area of spacer 1000 (e.g., relative to the surface area of bone plate 100) can advantageously reduce contact between the spacer assembly 1100 and the underlying bone 1200. The one or more spacers 1000 can be the primary points of contact between the spacer assembly 1100 and the bone surface, thereby preventing or reducing damage to the periosteum. This can promote improved healing of the bone since the periosteum contains fibroblasts and progenitor cells that develop osteoblasts for maintaining and healing bone.

In certain embodiments, as seen in FIG. 35, the upper and/or lower edges 1005 of the aperture 1004 can be radiused, and/or can comprise a chamfer or a bevel (e.g., the opening of the aperture in the surface 1008 can be countersunk). The edges of the body at the opening of the slot 1006 can also be angled to facilitate positioning the spacer around a fastener. In certain embodiments, the aperture 1004 can have a diameter that is the same as, greater than, or less than the width of the slot 1006. As best seen in FIG. 37, the aperture 1004 can have a first opening 1040 at a first end and a second opening 1042 at a second end. As shown, the second opening 1042 can have a greater diameter than the first opening 1040 such that the aperture 1004 tapers from the second opening 1042 to the first opening 1040. In other embodiments, the first opening 1040 can have a diameter greater than the second opening 1042. In still other embodiments, the openings 1040, 1042 can have equal or substantially equal diameters.

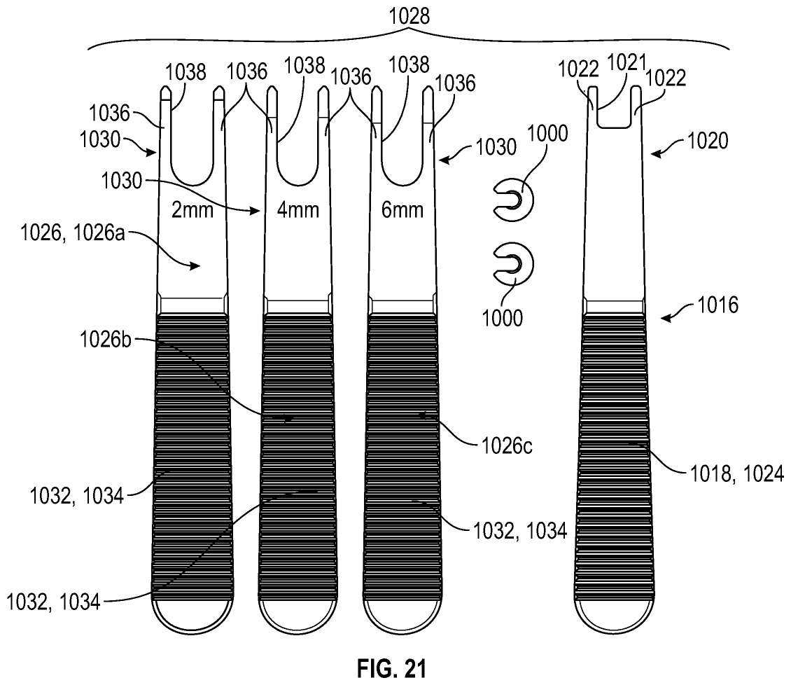

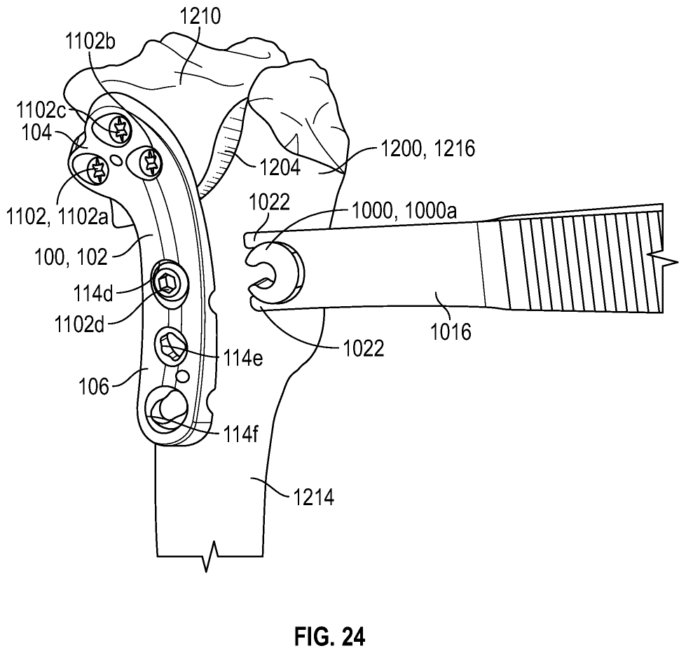

In some embodiments, such as the embodiment illustrated in FIGS. 18-19, the sidewall 1012 can comprise one or more slots 1014 extending radially inwardly toward the central aperture 1004. The slots 1014 can be configured to engage an inserter or positioning implement 1016 (FIG. 21), which can be used to position a spacer 1000 at a selected implantation site, e.g., as shown in FIG. 24. In the embodiment illustrated in FIG. 19, the spacer 1000 includes first and second opposing slots 1014a, 1014b, each of which has a substantially semi-circular shape. However, in other embodiments, the spacer 1000 can comprise any number of slots 1014 and the slots can have any shape configured to engage the positioning implement 1016. In still other embodiments, the sidewall 1012 can comprise a single continuous slot rather than the circumferentially offset slots 1014a and 1014b.

FIG. 21 illustrates a representative example of a positioning tool or implement 1016 that can be used to position a spacer at a treatment site, such as between a bone plate and a bone segment to space the bone plate away from the bone segment. As shown in FIG. 21, the positioning implement 1016 can have a handle 1018 and a tool head referred to as an engagement portion 1020. In some embodiments, the handle 1018 can comprise a gripping portion 1024, which can include a plurality of ridges or other surface texture, to facilitate gripping by the user. The engagement portion 1020 can be configured to engage the one or more slots 1014 of the spacer 1000 in order to position the spacer 1000 at a selected implantation site. For example, in the illustrated embodiment the engagement portion 1020 comprises a pair of prongs, tines, or projections 1022 (e.g., first and second projections) that define a recess 1021 between them. Each projection 1022 can be sized such that it can slide into a respective slot 1014 of the spacer 1000 so that the spacer 1000 is positioned within the recess 1021, as shown in FIG. 24.

Referring again to FIG. 19, the spacer 1000 can have a selected thickness T such that when the spacer assembly 1100 is implanted at an osteotomy, the selected offset between the upper and lower portions of the bone is created to provide biomechanical alignment similarly to the spacer assembly 300 described previously, and similarly to a stepped plate. In some embodiments, the thickness T of the spacer can be from 1 mm to 10 mm, from 1 mm to 6 mm, from 2 mm to 6 mm In some embodiments, the thickness T can be 1 mm, 2 mm, 3 mm, 4 mm, 5 mm, 6 mm, 7 mm, 8 mm, 9 mm, or 10 mm.

As mentioned above, and as shown in FIG. 20, one or more spacers 1000 (e.g., two spacers 1000a and 1000b in the illustrated embodiment) can be used with a bone plate (e.g., bone plate 100) to form a spacer assembly 1100. The spacer assembly 1100 can be configured to offset an upper portion 1202 of the bone 1200 (e.g., above or proximal of an osteotomy 1204 in the orientation shown in FIG. 20) from a lower portion 1206 of the bone (e.g., below or distal of the osteotomy 1204 in the orientation shown in FIG. 20). Each spacer 1000 can be disposed around a respective fastener 1102 that extends through the bone plate 100 such that the spacer 1000 is disposed between the bone plate 100 and the outer surface 1208 of the bone (also referred to as the cortex). In other embodiments, only one spacer 1000 can be used, or a greater number of spacers 1000 can be used (e.g., corresponding to a number of fasteners extending through the bone plate).

In the embodiment illustrated in FIG. 20, both spacers 1000a, 1000b have the same thickness T. However, in other embodiments, the spacers 1000 can have differing thicknesses. For example, spacer 1000a can have a thickness greater than spacer 1000b (or vice versa) to adjust the angle of the bone plate 100 relative to a longitudinal axis extending through the bone 1200.

In some embodiments, the spacer(s) 1000 can be made of any biocompatible material such as, for example, biocompatible metal (e.g., stainless steel, titanium, cobalt-chromium, and/or tantalum, etc.), biocompatible polymer (e.g., polyether ether ketone (PEEK)), and combinations thereof. In some embodiments, the spacer(s) 1000 can comprise any of various biocompatible and/or bioresorbable materials. For example, in some embodiments, the spacer can be configured to be naturally resorbed or dissolved by the body after a period of time has elapsed sufficient to allow the osteotomy to heal. In still other embodiments, the spacer can comprise a combination of non-resorbable biomaterials and bioresorbable materials such that only portions of the spacer can be dissolved.

As mentioned previously, it should be understood that the bone plates, spacers, spacer assemblies, and methods described herein are applicable to any long bones in canids, as well as in other mammals including felines and humans. More specifically, the ability to adjust the biomechanical alignment of a bone along multiple axes can allow the spacer assembly 1100 to be used with a variety of long bones and a variety of osteotomy procedures and/or fractures of those bones. The spacer assemblies described herein can also be used on multiple sides of the body, and at the proximal and distal ends of bones without significant modification, contrary to known bone plates.

In use, as shown in FIG. 20, the first spacer 1000a can be aligned with the first compression anchor opening 114d in the bone plate 100 and the second spacer 1000b can be aligned with the second compression anchor opening 114f. The spacers 1000 can be disposed between the lower surface 126 (FIG. 3) of the second end portion 106 of the bone plate 100 and the bone such that the second surface 1010 of each spacer 1000 contacts the bone 1200 in lieu of the second end portion 106 of the bone plate 100. In some embodiments, as shown, the one or more spacers 1000 can be coupled to the second end portion 106 of the bone plate 100, resulting in an increased thickness portion which can be configured to achieve a specified offset between the upper and lower bone segments 1202, 1206. In other embodiments, the spacers 1000 can be coupled to the first end portion 104 of the bone plate 100.

The spacer(s) 1000 can be coupled to the bone plate 100 during an implantation procedure, e.g., by positioning a spacer 1000 around a fastener 1102 extending through the bone plate 100 and into the native bone 1200. The fasteners 1102 can be for example, locking screws, non-locking screws, compression screws, or combinations thereof, which are configured to be driven into bone. In some embodiments, the bone plate 100 can comprise one or more grooves or recesses on the lower surface 126 in which the spacer(s) 1000 can be disposed. The grooves can, for example, be V-shaped grooves that lock the spacer 10 in place. In certain embodiments, the lower surface 126 of the bone plate can be curved in the manner of a dome along a portion or all of its length. The curvature of the lower surface of the bone plate can correspond to the curvature of the surfaces 1008 of the spacers so that the curved underside of the bone plate receives the spacers and prevents them from moving relative to the bone plate. In other embodiments, the bone plate 100 can have a catching or locking element that holds the spacer 1000 in place against the bone plate 100.

In some embodiments, the spacer assembly 1100 can be used in combination with one or more locking bone screws, which can provide a locking feature in the head of the screw that locks or engages the screw with the bone plate 100 and/or respective spacer 1000 when the screw is inserted into the bone. In other embodiments, the spacer(s) 1000 and bone plate 100 can be coupled together using, for example, welding, adhesives, or mechanical fasteners apart from those that extend fully through the assembly.

Referring to FIG. 21, in some embodiments, one or more spacers 1000 can be packaged and/or sold with a positioning implement 1016 and/or one or more sizing implements 1026 (or any combination of these elements) to form an assembly or kit 1028 packaged together for delivery to the end user. In some embodiments, the kit 1028 can further include a bone plate, such as bone plate 100, along with any combination of the previously recited elements. An exemplary kit 1028 comprising two spacers 1000, a positioning implement 1016, and three sizing implements 1026 (e.g., a 2 mm sizing implement 1026a, a 4 mm sizing implement 1026b, and a 6 mm sizing implement 1026c), is shown in FIG. 21.

The sizing implements 1026 can be used to determine a selected size for the spacer(s) 1000 to affect a desired offset of the bone for biomechanical alignment. Each sizing implement can comprise an engagement portion 1030, configured to be inserted between the bone plate (e.g., bone plate 100) and the bone 1200, e.g., as shown in FIG. 23, and a handle 1032. The engagement portion 1030 can comprise a pair of prongs or projections 1036 defining a recess 1038 between them. Such a configuration allows the engagement portion 1030 to be disposed around an already-inserted fastener, such that the fastener sits within the recess 1038. In some embodiments, the handle 1032 can comprise a gripping portion 1034, such as a plurality of ridges, to facilitate gripping by the user.

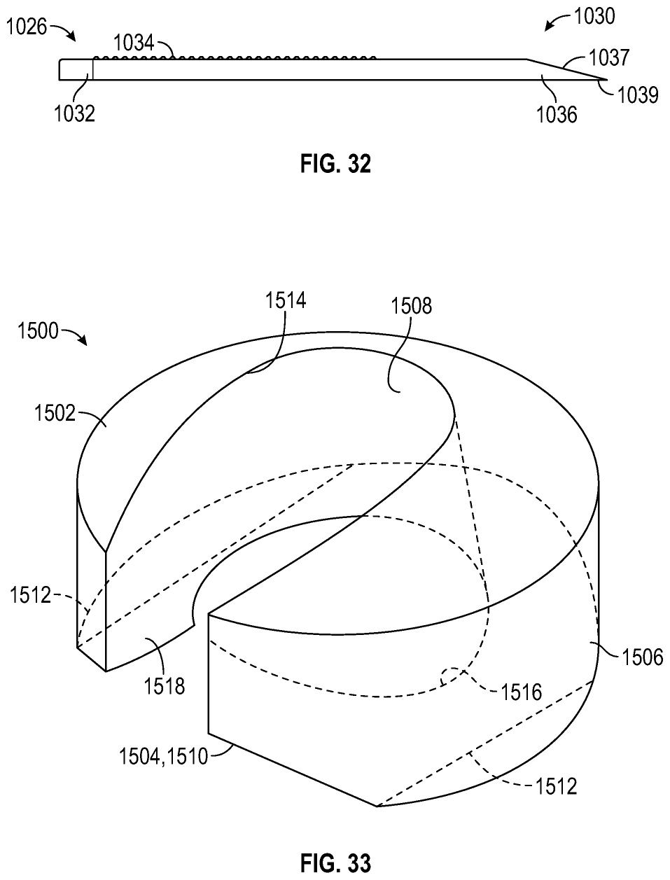

As shown in FIG. 32, each projection 1036 can comprise a tapered, chamfered, or ramped portion 1037 tapering from a first thickness at the free end 1039 to a second, greater thickness adjacent the handle 1032. Such a configuration advantageously allows the projections to be easily slid between the bone plate 100 and the native bone such that the spacing between the bone plate 100 and the native bone is increased as the engagement portion is advanced around the fastener. The second thickness can be, e.g., 2 mm, 4 mm, 6 mm, etc., and can correspond to the thickness of a respective spacer. In some embodiments, the sizing implements can be used sequentially to gradually and incrementally shift one bone portion relative to another, such as in a direction perpendicular or substantially perpendicular to the direction of advancement of the tool. For example, in a TPLO procedure, one or a plurality of sizing implements can be used to advance or displace the diaphysis of the tibia medially or laterally relative to the tibial plateau (and/or the tibial tubercle) by a selected distance prior to placement of the spacers.

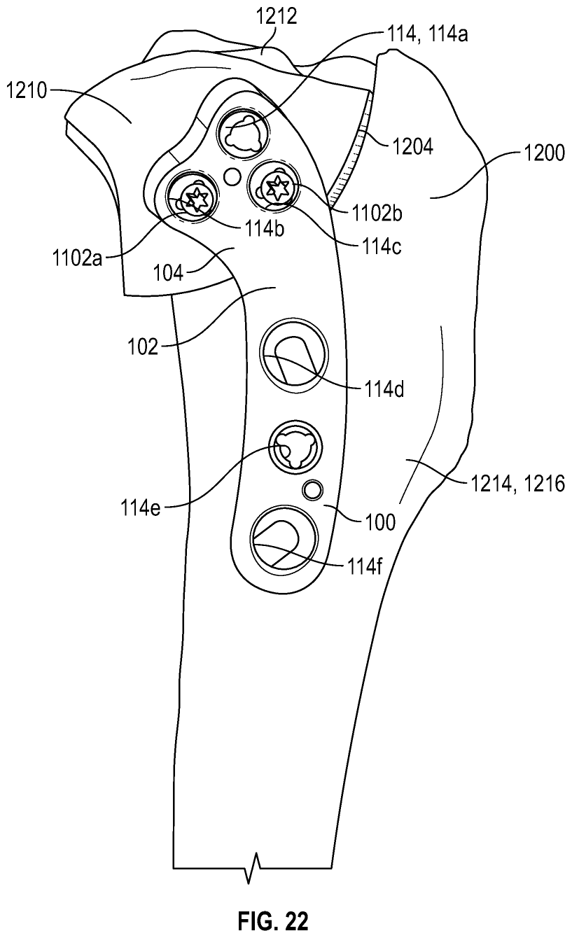

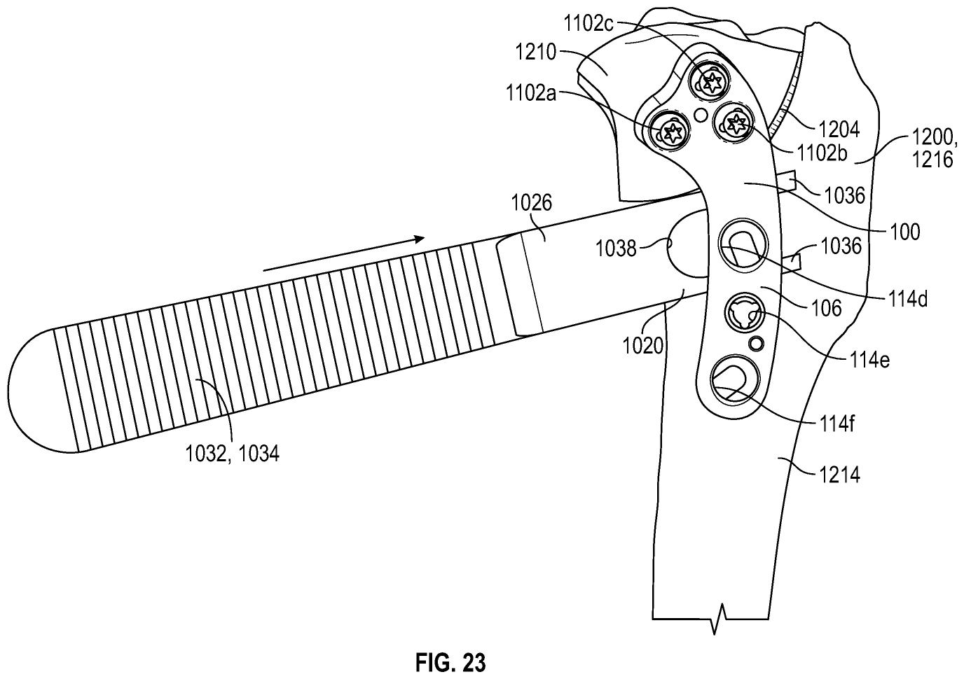

Referring to FIGS. 22-26, the spacer assembly 1100 can be implanted as part of a TPLO procedure on a native tibia 1200, as described in the following exemplary method. During a TPLO procedure it can be advantageous to shift the osteotomy medially using a spacer assembly, such as spacer assembly 1100, to realign the patella and mitigate or eliminate luxation of the patella out of the femoral trochlear groove.

For example, as shown in FIG. 22, a surgeon can cut an osteotomy 1204 through the head of the tibia 1200 such that the tibial plateau 1212 is separated from the main body/shaft 1214 of the tibia 1200, forming a proximal portion 1210 (e.g., a portion of the head portion including the tibial plateau 1212) and a distal portion 1216 (e.g., the main body/shaft 1214 of the tibia). In some embodiments, such as the illustrated embodiment, the osteotomy 1204 can be a curved cut. In other embodiments, the osteotomy can be a straight cut. Once the proximal portion 1210 has been separated it can be rotated until a selected angle of the tibial plateau 1212 is achieved. First and second fasteners 1102a, 1102b (e.g., locking screws), can be inserted into the cranial and caudal openings 114b and 114c of the bone plate 100, fixing the first end portion 104 of the bone plate 100 to the proximal portion 1210 of the tibia 1200. In some embodiments, as shown in FIGS. 23-24, a third fastener 1102c (e.g., a locking screw) can be inserted into opening 114a of the first end portion 104.

As shown in FIG. 23, one or more sizing implements 1026 can be disposed between the bone plate 100 and the distal portion 1216 of the tibia. The sizing implement 1026 can displace (e.g., transpose) the proximal portion 1210 of the tibia laterally (e.g., into the plane of the page in FIG. 23) by a selected amount depending on the sizing implement selected. In certain embodiments, the surgeon can transpose the bone portions in increments, for example by beginning with a relatively thin implement 1026 (e.g., 2 mm thickness), then using one or a series of thicker implements until a selected offset is achieved. The surgeon can then run the knee joint through a range of motion while assessing the potential for patellar luxation. If patellar luxation has been satisfactorily addressed, a spacer 1000 having a thickness corresponding to the selected lateral offset can be selected for implantation. For example, in the illustrated embodiment a 4 mm sizing implement is used, so a 4 mm spacer 1000 can be selected for implantation.

Referring to FIG. 24, a fastener 1102d (e.g., a compression screw) can be inserted into the proximal opening 114d in the second end portion 106 of the bone plate 100. The fourth fastener 1102d can be driven part way into the bone such that the bone plate 100 can slide medially along the shaft of the fastener. A first selected spacer 1000a (e.g., a 4 mm spacer) can then be loaded into the positioning implement 1016, as described previously. So loaded, the positioning implement 1016 can be used to position the spacer 1000a around the fastener 1102d such that the spacer 1000a is disposed between the bone plate 100 and the bone 1200. For example, the spacer can be advanced around the shaft of the screw in a direction perpendicular or substantially perpendicular to the longitudinal axis of the screw so that the screw slides through the slot 1006 and into the central bore 1004. In some embodiments, the sizing implement 1026 can still be in place while the spacer 1000a is inserted.

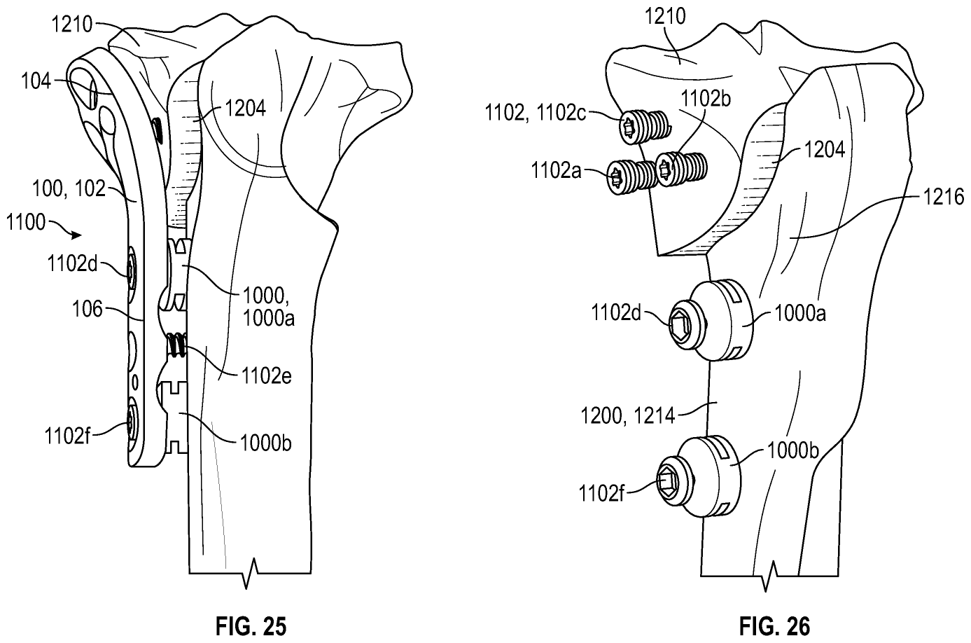

As shown in FIG. 25, a fastener 1102f (e.g., a compression screw) can be inserted into the distal opening 114f in the second end portion 106. A second selected spacer 1000b (e.g., a 4 mm spacer) can be loaded into the positioning implement 1016 and positioned around the fastener 1102f between the bone plate 100 and the bone 1200. FIG. 26 shows the spacer assembly 1100 without the bone plate 100 for purposes of illustration. The fastener 1102f can then be tightened, securing the second spacer 1000b in place. The fastener 1102d can then subsequently be tightened, securing the first spacer 1000a in place. An additional fastener 1102e (e.g., a locking screw) can be inserted into the opening 114e in the second end portion 106 which can aid in stabilization of the spacer assembly 1100.

In some embodiments, in lieu of spacers 1000, the sizing implement 1026 can be used to determine the selected spacing of the second end portion 106 of the bone plate 100 relative to the bone 1200, and one or more locking screws can be used to maintain the selected spacing (also referred to as “setting” or “fixing” the height/offset of the bone plate). For example, one or a plurality of locking bone screw with threaded heads can be inserted through openings in the second portion of the bone plate and driven into the bone by a selected distance. The threaded heads of the bone screws can then be tightened to engage the threads in the screw holes of the bone plate. This can immobilize the bone plate relative to the screw and relative to the bone, thereby setting the bone plate at a specified distance above the bone. In some such embodiments, the spacing between the bone plate and native bone can be achieved using the bone screws only and without the insertion of one or more spacers. However, in other embodiments, one or more spacers can then be inserted between the bone plate and the bone, and the bone screws tightened as needed to fix the assembly in place.

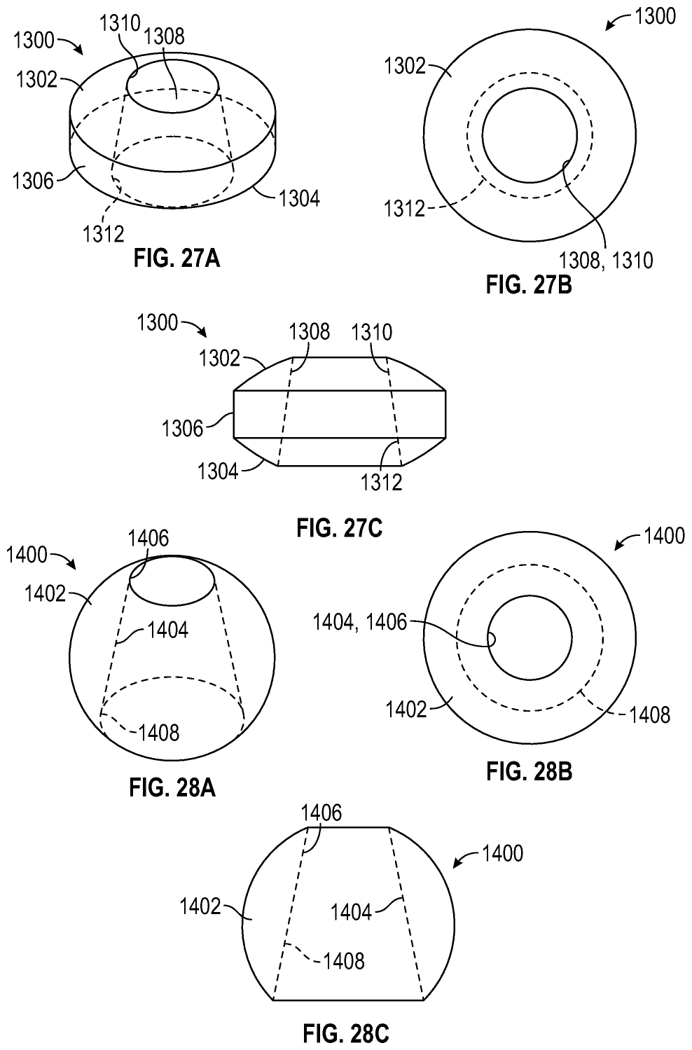

FIGS. 27A-31C illustrate various other embodiments of spacer designs. Spacers 1300, 1400, 1500, 1600, and 1700 can be used in lieu of or in addition to spacers 1000. For example, FIGS. 27A-27C illustrate an embodiment of a spacer 1300 having a substantially toroidal shape. The spacer 1300 can have a first surface 1302, a second surface 1304, a side wall 1306, and a central aperture or inner bore 1308. As best seen in FIG. 27C, the first and second surfaces 1302, 1304 can have a curved, convex or domed shape. The inner bore 1308 can have a first opening 1310 at a first end and a second opening 1312 at a second end. As shown, the second opening 1312 can have a greater diameter than the first opening 1310 such that the inner bore 1308 tapers from the second opening 1312 to the first opening 1310. In other embodiments, the first opening 1310 can have a diameter greater than the second opening 1312. In still other embodiments, the openings 1310, 1312 can have equal or substantially equal diameters.

FIGS. 28A-28C illustrate another embodiment of a spacer 1400. Spacer 1400 can have a main body 1402 having a substantially spherical shape truncated by openings 1406 and 1408 in the manner of a frustum of a sphere. A central aperture or inner bore 1404 can extend through the main body. The inner bore 1404 can have a first opening 1406 at a first end and a second opening 1408 at a second end. As shown, the second opening 1408 can have a greater diameter than the first opening 1406 such that the inner bore 1404 tapers from the second opening 1408 to the first opening 1406. This can result in the main body 1402 comprising a greater proportion of the upper hemisphere than the lower hemisphere, as shown in FIG. 28C. In other embodiments, the first opening 1406 can have a diameter greater than the second opening 1408. In still other embodiments, the openings 1406, 1408 can have equal or substantially equal diameters.

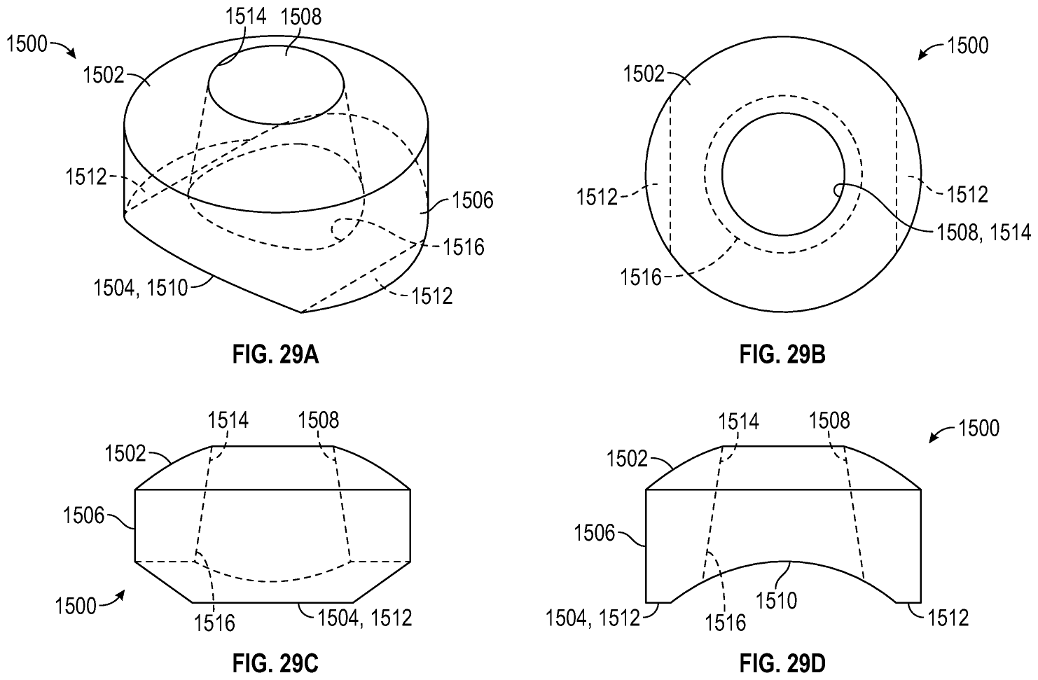

FIGS. 29A-29D illustrate another embodiment of a spacer 1500. Spacer 1500 can have a substantially toroidal shape, similar to spacer 1300, having a first surface 1502, a second surface 1504, a side wall 1506, and a central aperture or inner bore 1508. As best seen in FIGS. 29C-29D, the first surface 1502 can have a curved, convex, or domed shape. The second surface 1504 can comprise one or more recesses or cutouts 1510 extending into the thickness of the spacer. Referring to FIG. 29D, in the illustrated embodiment, the second surface 1504 comprises a cutout 1510 having a semicircular shape in cross-section such that the second surface 1504 has a substantially concave shape. Such a configuration advantageously provides that the spacer 1500 has a reduced surface area that contacts the surface of the bone. For example, the one or more cutouts 1510 can define one or more ledges 1512 that contact the bone when the spacer 1500 is implanted as part of a spacer assembly.

The inner bore 1508 can have a first opening 1514 at a first end and a second opening 1516 at a second end. As best seen in FIGS. 29C-29D, the second opening 1516 can have a greater diameter than the first opening 1514 such that the inner bore 1508 tapers from the second opening 1516 to the first opening 1514. In other embodiments, the first opening 1514 can have a diameter greater than the second opening 1516. In still other embodiments, the openings 1514, 1516 can have equal or substantially equal diameters.

As shown in FIG. 33, in some embodiments, the spacer 1500 can comprise a cutout or slot 1518 similar to slot 1006 of spacer 1000. The slot 1518 can be configured (e.g., sized and shaped) to receive a fastener such that the spacer 1500 can be disposed around a fastener, such as around the shaft of a screw, by sliding the fastener through the slot 1518. For example, as shown in FIG. 33, the slot 1518 can extend from the inner bore 1508 to a radially outer edge of the spacer 1500. In some embodiments, spacers 1300, 1400, 1600, and 1700 can each comprise a similar slot extending from the inner bore to a radially outer edge of the spacer.

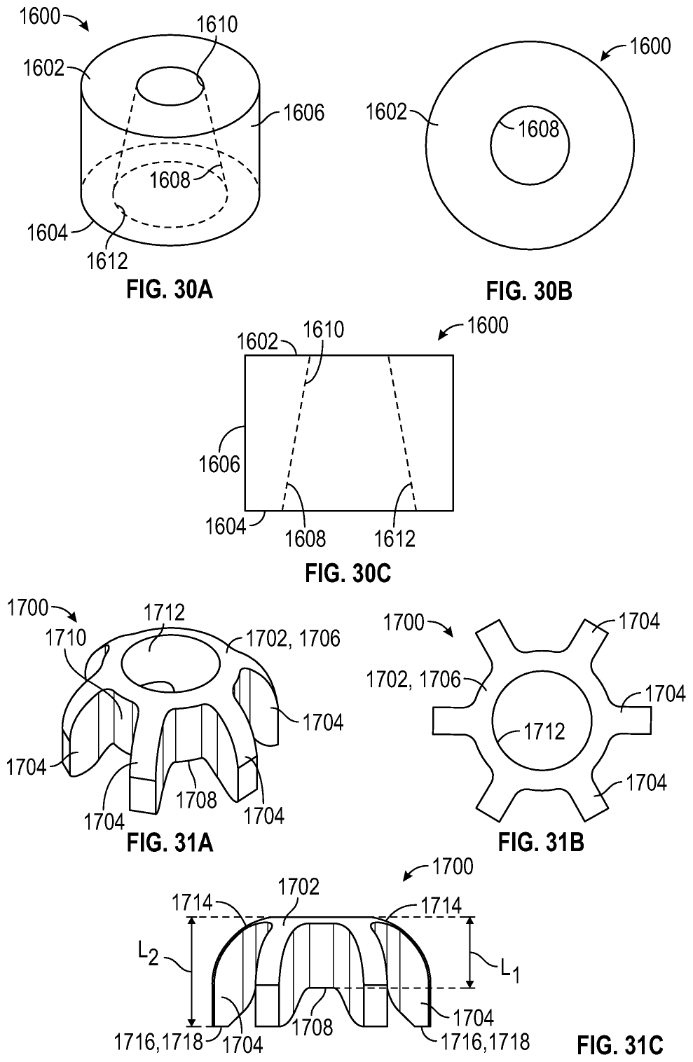

FIGS. 30A-30B illustrate yet another embodiment of a spacer 1600. Spacer 1600 can have a substantially cylindrical shape and can include a first surface 1602, a second surface 1604, a side wall 1606, and an inner bore 1608 extending through the spacer 1600. The inner bore 1608 can have a first opening 1610 at a first end and a second opening 1612 at a second end. As best seen in FIG. 30C, the second opening 1612 can have a greater diameter than the first opening 1610 such that the inner bore 1608 tapers from the second opening 1612 to the first opening 1610 resulting in a frustoconical inner volume. In other embodiments, the first opening 1610 can have a diameter greater than the second opening 1612. In still other embodiments, the openings 1610, 1612 can have equal or substantially equal diameters.

FIGS. 31A-31C illustrate still another embodiment of a spacer 1700. Spacer 1700 can have a main body 1702 having a cylindrical shape, and comprising one or more projections, flanges, ribs, or legs referred to hereafter as protrusions 1704 extending radially from the main body 1702. The main body 1702 can comprise a first surface 1706, a second surface 1708, a side wall 1710, and an inner bore 1712.

In the illustrated embodiment, the spacer 1700 comprises six protrusions 1704, however, in other embodiments, the spacer 1700 can comprise any number of protrusions 1704, for example, one, two, three, four, five, seven, eight, nine, or ten protrusions. In the illustrated embodiment, the protrusions 1704 are equally spaced from one another, but in other embodiments, the protrusions 1704 can be arranged with any spacing between them.

In some embodiments, such as the embodiment shown in FIGS. 31A-31C, the protrusions 1704 can have a first, curved surface portion 1714, and a second surface portion 1716 comprising a ledge 1718 positioned to contact a surface of the bone when the spacer 1700 is implanted. Referring to FIG. 31C, the main body 1702 can have a length along the longitudinal axis L₁ and the protrusions can have a length along the longitudinal axis L₂, greater than the first length L₁, such that a portion of the protrusions 1704 extends past the second surface 1708 of the main body 1702. Such a configuration advantageously provides that the spacer 1700 has a reduced surface area that contacts the surface of the bone because only the ledges 1718 contact the bone.

In the embodiments illustrated in FIGS. 27A-31C, the spacers can be implanted using a method similar to the method described previously for spacer 1000, except that spacers 1300-1700 can be disposed on a respective fastener 1102 prior to insertion of the fastener into the subject's bone. For example, the spacer can be positioned between the bone plate (e.g., bone plate 100) and the native bone and a fastener 1102 can be disposed through respective openings in the bone plate and spacer.

The features of any of the spacer members discussed herein can be used with any other spacer members, except where structurally impossible. For example, the projections 1704 shown in FIGS. 31A-31C can be used in combination with spacer 1000, the slots 1014 can be used in combination with any of the spacers 1300-1700, the tapered inner bore shown in FIGS. 27A-31C can be used in combination with spacer 1000, the positioning and sizing implements shown in FIG. 21 can be used in combination with any of the spacers 1300-1700, etc. Additionally, any or all of the spacers 1300-1700 can comprise a slot or cutout similar to the slot 1006 extending from the radially outer edge and communicating with the central bore/aperture to allow the spacers to be positioned around the shaft of a bone screw as described above. Still further, any or all of the spacers 1300-1700 can comprise one or more radially-inwardly extending slots similar to slots 1014 to allow the spacers to be mounted on a positioning tool for positioning around the shaft of a bone screw as described above.

In any of the representative orthopedic procedures described herein, multiple spacer members of the same or different thicknesses can be combined/stacked together to achieve the specified offset.

As used herein, the term “long bone” refers to a bone that has a length dimension greater than its diameter or width, and including, for example, the tibia, the femur, and the humerus.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An orthopedic spacer, comprising:
a curved body including a first end portion, a second end portion, and a central portion, the first and second end portions being laterally offset relative to the central portion, the curved body having a first surface and a second surface, the first surface comprising a plurality of convex protrusions and the second surface comprising a plurality of scallop-shaped recesses;
a set of first openings defined in the body along a first arc, the first arc having a first radius, the first openings having a first diameter, each first opening configured to receive an anchoring element to anchor the spacer to an implantation site; and
a set of second openings defined in the body along a second arc, the second arc having a second radius different from the first radius, each second opening having a second diameter smaller than the first diameter of the first openings.

Example 2. The orthopedic spacer of any example herein, particularly example 1, wherein each second opening is disposed on a first side of a longitudinal axis extending through the spacer.

Example 3. The orthopedic spacer of any example herein, particularly any one of examples 1-2, further comprising a third opening extending through the central portion of the curved body.

Example 4. The orthopedic spacer of any example herein, particularly example 3, further comprising a recessed portion surrounding the third opening, the recessed portion extending into the first surface of the body.

Example 5. The orthopedic spacer of any example herein, particularly any one of examples 1-4, wherein the curved body comprises a first side portion and a second side portion and wherein each side portion comprises a plurality of scallop-shaped recesses.

Example 6. The orthopedic spacer of any example herein, particularly any one of examples 1-5, wherein the spacer comprises one or more of stainless steel, titanium, and polyether ether ketone (PEEK).

Example 7. The orthopedic spacer of any example herein, particularly any one of examples 1-6, wherein a thickness of the spacer is between 1 mm and 10 mm.

Example 8. A method, comprising: securing the orthopedic spacer of any of any example herein, particularly examples 1-7 to a bone.

Example 9. An orthopedic spacer assembly, comprising:
a spacer comprising a curved body including a first end portion and a second end portion both laterally offset relative to a longitudinal axis of the spacer, and a set of openings defined in the body along a first arc having a first radius;
a bone plate including a first end portion and a second end portion, the first end portion being laterally offset relative to the second end portion, a first plurality of anchor openings defined by the first end portion and a second plurality of anchor openings defined by the second end portion; and
wherein the spacer is coupled to the second end portion of the bone plate such that the set of openings in the spacer align with the second plurality of anchor openings in the bone plate.

Example 10. The spacer assembly of any example herein, particularly example 9, wherein the set of openings in the spacer is a set of first openings and wherein the spacer further comprises a set of second openings defined in the body along a second arc having a second radius.

Example 11. The spacer assembly of any example herein, particularly any one of examples 9-10, wherein each second opening has a diameter smaller than a diameter of each first opening.

Example 12. The spacer assembly of any example herein, particularly any one of examples 9-11, wherein the spacer comprises a first surface and a second surface, the second surface comprising a plurality of recesses defining a scalloped shape.

Example 13. The spacer assembly of any example herein, particularly any one of examples 9-12, wherein the first surface of the spacer comprises a plurality of convex protrusions.

Example 14. The spacer assembly of any example herein, particularly example 13, wherein a first surface of the bone plate comprises a curved shape, such that when the spacer is coupled to the bone plate the curved shape mates with the convex protrusions.

Example 15. The spacer assembly of any example herein, particularly any one of examples 11-14, wherein the first surface of the spacer is positioned adjacent the bone plate and wherein the second surface is configured to be positioned adjacent a native bone.

Example 16. The spacer assembly of any example herein, particularly any one of examples 9-15, wherein a thickness of the spacer is between 1 mm and 10 mm.

Example 17. The spacer assembly of any example herein, particularly any one of examples 10-16, wherein the first radius and second radius are different.

Example 18. A method, comprising: securing the orthopedic spacer assembly of any example herein, particularly any one of examples 9-17 to a bone.

Example 19. The spacer assembly of any example herein, particularly any one of examples 9-18, wherein the spacer assembly is configured to be implanted at a selected implantation site to shift at least one of a proximal portion and a distal portion of a tibia such that a longitudinal axis of the proximal portion is offset from a longitudinal axis of the distal portion.

Example 20. A spacer assembly, comprising:
a bone plate comprising:
a curved elongated body including a first end portion laterally offset from a second end portion, the first end portion comprising a first lobe defining a proximal screw hole, a second lobe defining a distal screw hole, and a third screw hole intermediate the proximal and distal screw holes and laterally offset from the proximal and distal screw holes, the second end portion comprising a first screw hole and a second screw hole; and
a spacer coupled to the second end portion of the bone plate, the spacer comprising:
a body including a first end portion, a second end portion, and a central portion, the first and second end portions being laterally offset relative to the central portion, the body having a first surface and a second surface, the first surface disposed adjacent the bone plate and comprising a plurality of convex protrusions and the second surface comprising a plurality of recesses defining a scalloped shape, and
a first opening positioned to align with the first screw hole and a second opening positioned to align with the second screw hole, the first and second openings defined in the body along a first arc having a first radius.

Example 21. A system comprising a spacer according to any of the embodiments described herein in combination with any of the bone plates described herein.

Example 22. A method, comprising:
creating an osteotomy in a long bone to create a first bone portion and a second bone portion;
offsetting the first and second bone portions such that the first and second bone portions are in an offset position; and
securing the first and second bone portions in the offset position with a bone plate and one or more orthopedic spacer members according to any of the embodiments described herein.

Example 23. A kit, comprising:
a bone plate according to any of the embodiments described herein; and
at least one orthopedic spacer member according to any of the embodiments described herein.

Example 24. An orthopedic spacer, comprising:
a main body having an overall c-shape and comprising a central bore extending through a thickness of the main body;
wherein the spacer configured to be disposed between a bone plate and an outer surface of a subject's bone such that the central aperture of the spacer aligns with an anchor opening of the bone plate and such that a fastener extending through the anchor opening extends through the central bore.

Example 25. The spacer of any example herein, particularly example 24, wherein the spacer comprises a first surface and a second surface separated by a sidewall, and wherein the first surface has a convex shape.

Example 26. The spacer of any example herein particularly any one of examples 24-25, wherein the sidewall comprises one or more slots extending radially inwardly toward the central aperture.

Example 27. A kit, comprising:
at least one orthopedic spacer member according to any of the embodiments described herein;
at least one sizing implement comprising a handle and an engagement portion, the engagement portion configured to be positioned between a bone plate and a subject's bone to determine a desired offset of a first portion of the bone from a second portion of the bone; and
a positioning implement configured to engage the orthopedic spacer to position the spacer at a desired location.

Example 28. An orthopedic spacer assembly, comprising:
a bone plate including a first end portion configured to couple a first bone portion defined by an osteotomy and a second end portion configured to couple a second bone portion defined by the osteotomy, the first end portion and the second end portion each defining a plurality of anchor openings;
a spacer having a central bore extending through a thickness of the spacer such that the spacer can be disposed at least partially around a fastener extending through a respective anchor opening in the second end portion; and
wherein the spacer is sized to laterally offset the first and second bone portions such that the first bone portion defined by the osteotomy is offset from a longitudinal axis of the bone.

Example 29. An orthopedic spacer assembly, comprising:
a bone plate including a first end portion and a second end portion, the first end portion and the second end portion each defining a plurality of anchor openings;
a spacer having a central bore extending through a thickness of the spacer such that the spacer can be disposed at least partially around a fastener when a fastener is inserted through a respective anchor opening in the bone plate; and
wherein the spacer is sized to laterally offset the first and second bone portions such that the first bone portion defined by the osteotomy is offset from a longitudinal axis of the bone.

Example 30. The orthopedic spacer assembly of any example herein, particularly example 29, wherein the spacer comprises a first surface and a second surface separated by a sidewall, and wherein the first surface has a convex shape.

Example 31. The orthopedic spacer assembly of any example herein, particularly example 30, wherein the sidewall is circular.

Example 32. The orthopedic spacer assembly of any example herein, particularly example 30, wherein the sidewall comprises a slot extending radially inwardly toward the central bore, and wherein the slot is configured to engage a positioning tool configured to position the spacer between the bone plate and the second bone portion.

Example 33. The orthopedic spacer assembly of any example herein, particularly example 32, wherein:
the slot is a first slot; and
the sidewall further comprises a second slot circumferentially offset from the first slot around the body of the spacer from the first slot.

Example 34. The orthopedic spacer assembly of any example herein, particularly example 30, wherein the first surface of the spacer is configured to be positioned adjacent the bone plate and wherein the second surface is configured to be positioned against a bone portion.

Example 35. The orthopedic spacer assembly of any example herein, particularly any one of examples 29-34, wherein the central bore tapers from a first opening having a first diameter to a second opening having a second diameter greater than the first diameter.

Example 36. The orthopedic spacer assembly of any example herein, particularly any one of examples 29-35, wherein the spacer comprises a cutout extending from the central bore to a radially outer edge of the spacer.

Example 37. The orthopedic spacer assembly of any example herein, particularly any one of examples 29-36, wherein the spacer has a toroidal shape.

Example 38. The orthopedic spacer assembly of any example herein, particularly any one of examples 29-37, wherein the second surface of the spacer comprises one or more cutouts.

Example 39. The orthopedic spacer assembly of any example herein, particularly example 38, wherein the one or more cutouts define one or more ledges configured to contact the second bone portion.

Example 40. The orthopedic spacer assembly of any example herein, particularly any one of examples 29-39, wherein the spacer comprises one or more protrusions extending radially from the main body.

Example 41. The orthopedic spacer assembly of any example herein, particularly example 40, wherein the protrusions have a thickness greater than a thickness of the main body such that a portion of the protrusions extends past the second surface of the main body.

Example 42. The orthopedic spacer assembly of any example herein, particularly any one of examples 29-41, wherein a thickness of the spacer is between 1 mm and 10 mm.

In view of the many possible embodiments to which the principles of the disclosure may be applied, it should be recognized that the illustrated embodiments are only preferred examples and should not be taken as limiting the scope of the disclosure. Rather, the scope is at least as broad as the following claims. We therefore claim all that comes within the scope and spirit of these claims.

1. An orthopedic spacer assembly, comprising:
a bone plate including a first end portion and a second end portion, the second end portion defining a plurality of anchor openings; and
a spacer having a c-shaped body and comprising a central bore extending through a thickness of the spacer, the spacer configured to be disposed between the bone plate and a subject's bone such that the central bore of the spacer aligns with an anchor opening of the plurality of anchor openings.

2. The orthopedic spacer assembly of claim 1, wherein the spacer comprises a first surface and a second surface separated by a sidewall, and wherein the first surface has a convex shape.

3. The orthopedic spacer assembly of claim 2, wherein the side wall is circular.

4. The orthopedic spacer assembly of claim 2, wherein the sidewall comprises a slot extending radially inwardly toward the central bore, wherein the slot is configured to engage a positioning tool configured to position the spacer between the bone plate and a bone portion.

5. The orthopedic spacer assembly of claim 4, wherein:
the slot is a first slot; and
the side wall further comprises a second slot circumferentially offset from the first slot around the body of the spacer.

6. The orthopedic spacer assembly of claim 2, wherein the first surface of the spacer is configured to be positioned adjacent the bone plate and wherein the second surface is configured to be positioned against a bone portion.

7. The orthopedic spacer assembly of claim 1, wherein the central bore tapers from a first opening having a first diameter to a second opening having a second diameter greater than the first diameter.

8. The orthopedic spacer assembly of claim 1, wherein the spacer comprises an opening extending from the central bore to a radially outer edge of the spacer.

9. The orthopedic spacer assembly of claim 1, wherein a thickness of the spacer is between 1 mm and 10 mm.

10. The orthopedic spacer assembly of claim 1, wherein the spacer is a first spacer and the assembly further comprises a second spacer configured to be disposed between the bone plate and the subject's bone such that the central bore of the second spacer aligns with another of the anchor openings of the plurality of anchor openings.

11. The orthopedic spacer assembly claim 1, wherein the spacer is configured to be disposed at least partially around a fastener extending through the anchor opening.

12. The orthopedic spacer assembly of claim 1, wherein a second surface of the spacer comprises one or more cutouts.

13. An orthopedic spacer, comprising:
a curved main body having a longitudinal axis;
the main body having a convex first surface;
the main body having a second surface on the opposite side of the main body from the first surface along the longitudinal axis;
a first slot extending longitudinally through a thickness of the main body, and extending radially inwardly from an outer perimeter of the main body to a center of the main body such that the main body is C-shaped.

14. The orthopedic spacer of claim 13, wherein the main body further comprises a sidewall between the first surface and the second surface, and the sidewall comprises a second slot extending radially inwardly from an outer surface of the sidewall.

15. The orthopedic spacer of claim 14, wherein the sidewall further comprises a third slot circumferentially offset from the second slot around the main body of the spacer from the second slot.

16. The orthopedic spacer of claim 13, wherein the main body comprises chamfered surfaces at an open end of the first slot.

17. The orthopedic spacer of claim 13, wherein a closed end portion of the first slot at the center of the main body is countersunk.

18. A method, comprising:
creating an osteotomy in a long bone to create a first bone portion and a second bone portion;
securing a first end portion of a bone plate to the first bone portion;
offsetting the first and second bone portions such that the first and second bone portions are in an offset position relative to one another;
disposing one or more spacers between a second end portion of the bone plate and the second bone portion; and
securing the first and second bone portions in the offset position using one or more fasteners.

19. The method of claim 18, wherein offsetting the first and second bone portions from one another comprises inserting a sizing implement between a second end portion of the bone plate and the second bone portion.

20. The method of claim 18, wherein disposing one or more spacers between the bone plate and the second bone portion comprises mounting a spacer on a positioning implement, and positioning the spacer around a fastener extending through the bone plate and into the second bone portion using the positioning implement.
